(19) United States
(12) Patent Application Publication
(10) Pub. No.: US 2026/0053511 A1
(43) Pub. Date: Feb. 26, 2026

(54) SYSTEM AND METHOD FOR PERFORMING QUADRUPED UNICOMPARTMENTAL STIFLE ARTHROPLASTY

(71) Applicant: UNIVERSITY OF FLORIDA RESEARCH FOUNDATION, INCORPORATED, Gainesville, FL (US)

(72) Inventors: Scott Arthur BANKS, Gainesville, FL (US); Adam Henry BIEDRZYCKI, Gainesville, FL (US); Hongjia HE, Gainesville, FL (US); Dario Gonzalo SAAVEDRA, Gainesville, FL (US); Trevor PEREZ, Gainesville, FL (US); Ninoshka SUTCLIFFE, Gainesville, FL (US); Katherine ZARONIAS, Gainesville, FL (US); Joshua TSE, Gainesville, FL (US); Tyler SHROLL, Gainesville, FL (US)

(21) Appl. No.: 19/102,658

(22) PCT Filed: Aug. 9, 2023
(86) PCT No.: PCT/US2023/029820

§ 371 Date: Feb. 10, 2025

Related U.S. Application Data
(60) Provisional application No. 63/370,810, filed on Aug. 9, 2022.

(51) Int. Cl. A61B 17/17; A61F 2/30; A61F 2/38; A61F 2/46

(52) U.S. Cl. CPC A61B 17/1764; A61F 2/30749; A61F 2/389; A61F 2/4684; A61F 2002/307; A61F 2002/3895

(57) ABSTRACT

A method is provided for performing a quadruped unicompartmental stifle arthroplasty. The method includes cutting a plateau of a tibia to form first cut surfaces. The method also includes cutting a medial condyle of a femur to form second cut surfaces. The method also includes aligning openings in a first trial device with the first cut surfaces and forming first holes in the first cut surfaces by passing a drill through the openings in the first trial device. The method also includes aligning openings in a second trial device with the second cut surfaces and forming second holes in the second cut surfaces by passing a drill through the openings in the second trial device. The method also includes mounting a first implant device to the first cut surfaces and mounting a second implant device to the second cut surfaces. A system is also provided for performing the method.

### CROSS-REFERENCE TO RELATED APPLICATIONS

This application claims benefit of U.S. Provisional Application No. 63/370,810, filed Aug. 9, 2022 under 35 U.S.C. § 119(e).

### BACKGROUND

Knee replacement is a surgical procedure that decreases pain and improves the quality of life in many patients with severe arthritis of the knees. Typically patients undergo this surgery after non-operative treatments (such as activity modification medications knee injections or walking with a cane) have failed to provide relief of arthritic symptoms. Broadly speaking there are two types of knee replacements: total knee replacements and unicompartmental (or partial) knee replacements. Partial (unicompartmental) knee replacements have been around for decades and offer excellent clinical results just like total knee replacements.

### SUMMARY

Techniques are provided for performing a unicompartmental stifle arthroplasty (UKA) on a mammal (e.g. non-human mammal, such as a quadruped). In one embodiment, the quadruped is a horse.

FIGS. 1A and 1B are images that illustrate an example of a front view of a stifle or knee joint 100. The knee joint 100 is a junction of the femur 102 and tibia 104. The patella 106 covers a front of the knee joint 100. As shown in FIG. 1B, behind the patella 106 a base of the femur 102 at the knee joint 100 includes femoral condyles 108. As also shown in FIG. 1B, a top of the tibia 104 is called the tibial plateau 109. FIG. 1C depicts different compartments of the knee joint 100 where arthritis may necessitate a knee replacement. As appreciated by one of ordinary skill in the art, when arthritis is present in each of the lateral compartment 110, patellofemoral compartment 112 and medial compartment 114, a total knee replacement may be necessary. However, if arthritis is only present in one of the compartments (e.g. medial compartment 114), then only a partial or unicompartmental knee replacement may be necessary. FIG. 1D depicts cavities 116, 118 respectively formed in the medial condyle 108 and tibial plateau 109 after which implants 120, 122 (and insert 123) are secured within these cavities 116, 118 to perform a partial knee replacement of the medial compartment 114. For purposes of this description, a partial or unicompartmental knee replacement of the medial compartment 114 is discussed, however the embodiments of the invention are not limited to a partial or unicompartmental knee replacement of the medial compartment 114 and can be utilized for a partial or unicompartmental knee replacement of the lateral compartment 110 or patellofemoral compartment 112.

The inventors of the present invention recognized that while unicompartmental knee replacements, such as depicted in FIG. 1E, are available for human subjects, such a procedure is not well known for non-human subjects (e.g. horses). However, the inventors of the present invention recognized that this procedure could provide much therapeutic benefit to such non-human subjects, were it available. Thus, the inventors developed the system and method disclosed herein for performing unicompartmental knee arthroplasty on non-human subjects (e.g. horse) which includes numerous design specifics that are tailored to the anatomical differences between such non-human subjects and human subjects.

In a first set of embodiments, a system is provided for performing a unicompartmental knee arthroplasty in a knee of a mammal. The system includes a first cutting device configured to cut a portion of a plateau of a tibia to form a plurality of first cut surfaces in the tibial plateau. The system also includes a second cutting device configured to cut a portion of a medial condyle of a femur to form a plurality of second cut surfaces of the medial condyle. The system also includes a first trial device defining a plurality of openings to form a respective plurality of first holes in at least one of the first cut surfaces that are aligned with the plurality of openings. The system also includes a second trial device defining a plurality of openings to form a respective plurality of second holes in at least one of the second cut surfaces that are aligned with the plurality of openings. The system also includes a first implant device including a plurality of pegs configured to be inserted in the plurality of first holes to securely mount the first implant device to the first cut surfaces of the tibial plateau. The system also includes a second implant device including a plurality of pegs configured to be inserted in the plurality of second holes to securely mount the second implant device to the second cut surfaces of the medial condyle.

In a second set of embodiments, a method is provided for performing a unicompartmental knee arthroplasty in a knee of a mammal. The method includes cutting, with a first cutting device, a portion of a plateau of a tibia to form a plurality of first cut surfaces in the tibial plateau. The method also includes cutting, with a second cutting device, a portion of a medial condyle of a femur to form a plurality of second cut surfaces of the medial condyle. The method also includes aligning a plurality of openings in a first trial device with the first cut surfaces in the tibial plateau. The method also includes forming a respective plurality of first holes in the first cut surfaces based on passing a drill through the plurality of openings in the first trial device. The method also includes aligning a plurality of openings in a second trial device with the plurality of second cut surfaces of the medial condyle. The method also includes forming a respective plurality of second holes in the second cut surfaces based on passing a drill through the plurality of openings in the second trial device. The method also includes mounting a first implant device to the first cut surfaces of the tibial plateau based on inserting a plurality of pegs of the first implant device in the plurality of first holes. The method also includes mounting a second implant device to the second cut surfaces of the medial condyle based on inserting a plurality of pegs of the second implant device in the plurality of second holes.

Still other aspects, features, and advantages are readily apparent from the following detailed description, simply by illustrating a number of particular embodiments and implementations, including the best mode contemplated for carrying out the invention. Other embodiments are also capable of other and different features and advantages, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

Embodiments are illustrated by way of example, and not by way of limitation, in the figures of the accompanying drawings in which like reference numerals refer to similar elements and in which:

FIGS. 1A and 1B are images that illustrate an example of a front view of a knee joint;

FIG. 1C is an image that illustrates an example of a front view of different compartments of a knee joint;

FIG. 1D is an image that illustrates an example of a front view of cavities formed in the medial condyle and tibial plateau in performing unicompartmental knee arthroplasty;

FIG. 1E is an image that illustrates an example of a front view of implants mounted within the cavities of FIG. 1D;

FIG. 2 is a flow diagram that illustrates an example of a method for performing unicompartmental knee arthroplasty, according to one embodiment;

FIGS. 3A through 3E are images that illustrate an example of first cutting devices of a system that are used to perform a cutting step of the method of FIG. 2 to form cut surfaces in the tibial plateau, according to one embodiment;

FIGS. 4A through 4K are images that illustrate an example of the first cutting devices of FIGS. 3A through 3E being used to perform the cutting step to form the cut surfaces in the tibial plateau, according to one embodiment;

FIGS. 5A through 5D are images that illustrate an example of a second cutting device of a system that are used to perform a cutting step of the method of FIG. 2 to form a cut surface in the medial condyle, according to one embodiment;

FIGS. 6A through 6F are images that illustrate an example of the second cutting device of FIGS. 5A through 5D being used to perform the cutting step to form the cut surface in the medial condyle, according to one embodiment;

FIGS. 7A through 7E are images that illustrate an example of a second cutting device of a system that are used to perform a cutting step of the method of FIG. 2 to form additional cut surfaces in the medial condyle, according to one embodiment;

FIGS. 8A through 8D are images that illustrate an example of the second cutting device of FIGS. 7A through 7E being used to perform the cutting step to form the additional cut surfaces in the medial condyle, according to one embodiment;

FIGS. 9A through 9D are images that illustrate an example of trial devices of a system that are used to perform steps of the method of FIG. 2 to form respective holes in the cut surface in the tibial plateau and the cut surfaces in the medial condyle, according to one embodiment;

FIGS. 10A and 10B are images that illustrate an example of the trial devices of FIGS. 9A through 9D being used to form the respective holes in the cut surfaces of the tibial plateau and medial condyle, according to one embodiment;

FIGS. 11A through 11F are images that illustrate an example of implant devices of a system that are used to perform steps of the method of FIG. 2 to mount implant devices to the respective holes formed in the tibial plateau and medial condyle, according to one embodiment;

FIGS. 12A through 12D are images that illustrate an example of the implant devices of FIGS. 11A through 11F being used to mount the implant devices to the tibial plateau and medial condyle, according to one embodiment;

FIG. 13A is an image that illustrates an example of a tibial sizer used to determine an optimal size of a tibial insert to be used in performing steps of the method of FIG. 2, according to one embodiment;

FIG. 13B is an image that illustrates an example of a multi-use handle used to facilitate insertion and extraction of wedges in performing steps of the method of FIG. 2, according to one embodiment;

FIGS. 14A through 14D are images that illustrate an example of femoral implant design based on a mean shape generated by a statistic shape model of the femur, according to one embodiment;

FIGS. 15A and 15B are images that illustrate an example of tibial implant design based on a mean geometry generated by a statistic shape model of the tibia, according to one embodiment;

FIGS. 16A through 16C are images that illustrate an example of an assembly of the tibial implant and femoral implant in preparation for finite element (FE) models, according to one embodiment;

FIG. 17 is an image that illustrates an example of a meshed FE model with axial loading of a fixed tibial implant design, according to one embodiment;

FIGS. 18A and 18B are graphs that illustrates an example of peak contact pressure and peak Von Mises stress in a first group of tibial implants, according to one embodiment;

FIGS. 19A through 19D are images that illustrate an example of a comparison of plastic deformation and contact area at a maximum contact pressure in a first group of tibial implants, according to one embodiment;

FIGS. 20A and 20B are graphs that illustrates an example of peak contact pressure and peak Von Mises stress in a second group of tibial implants, according to one embodiment; and

FIGS. 20C and 20D are graphs that illustrates an example of peak contact pressure and peak Von Mises stress in a third group of tibial implants, according to one embodiment.

### DETAILED DESCRIPTION

A method and system are described for performing unicompartmental knee arthroplasty in a knee of a mammal. In the following description, for the purposes of explanation, numerous specific details are set forth in order to provide a thorough understanding of the present invention. It will be apparent, however, to one skilled in the art that the present invention may be practiced without these specific details. In other instances, well-known structures and devices are shown in block diagram form in order to avoid unnecessarily obscuring the present invention.

Some embodiments of the invention are described below in the context of performing unicompartmental knee arthroplasty in a knee of a non-human mammal (e.g. horse). However, the invention is not limited to this context. In other embodiments the invention is described in the context of performing unicompartmental knee arthroplasty in a knee of any mammal (e.g. other equids and ruminants, e.g., horses, donkeys, zebras, and cattle, sheep, giraffes, antelope and/or equine, bovine, canine and porcine.).

### 1. Overview

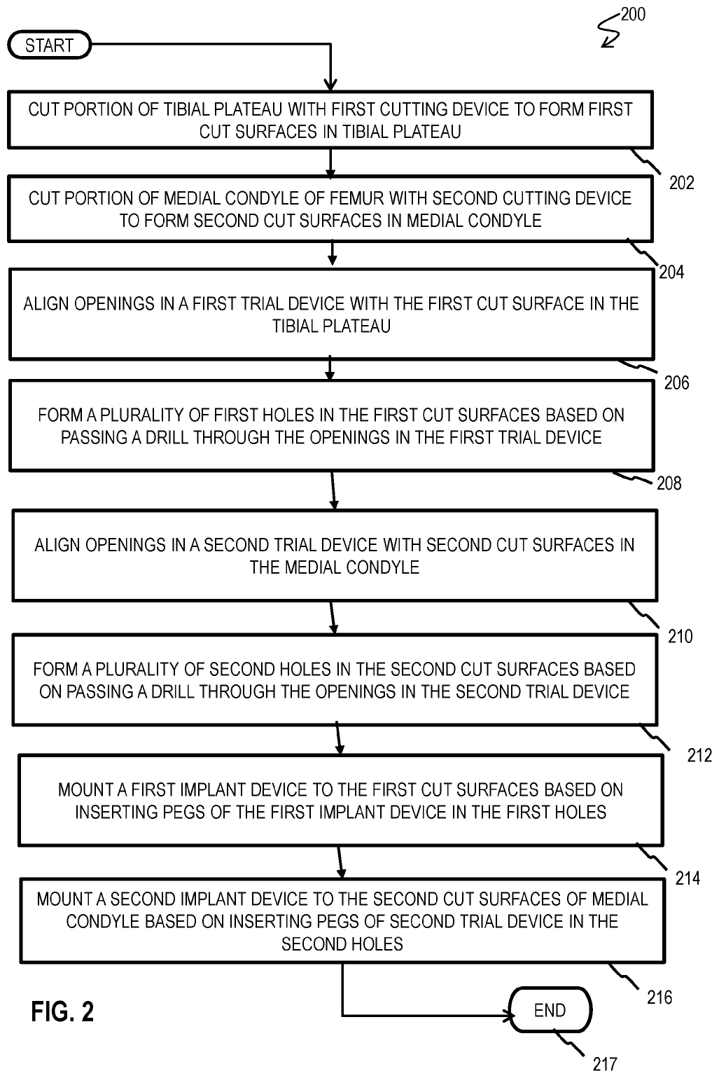

A method for performing unicompartmental kncc arthroplasty will now be discussed. In one embodiment, this method is used to performing unicompartmental knee arthroplasty on a non-human mammal (e.g. horse). Each step of the method will be discussed separately, along with one or more components of a kit or system that is utilized in performing each step of the method. FIG. 2 is a flow diagram that illustrates an example of a method 200 for performing unicompartmental knee arthroplasty, according to one embodiment. Although steps are depicted in FIG. 2 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

### 1.1 Cutting the Tibia

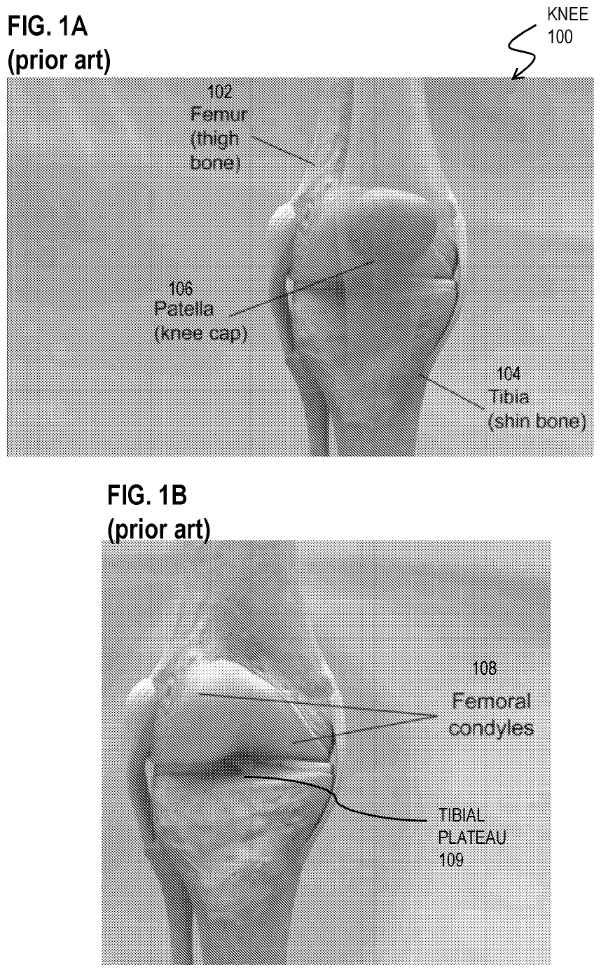

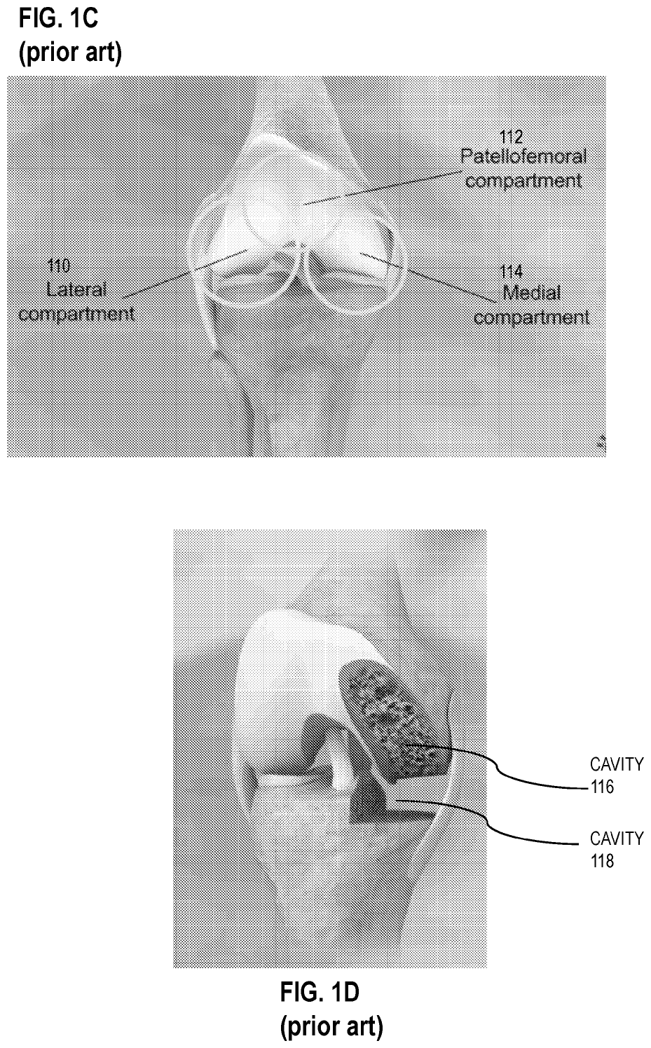

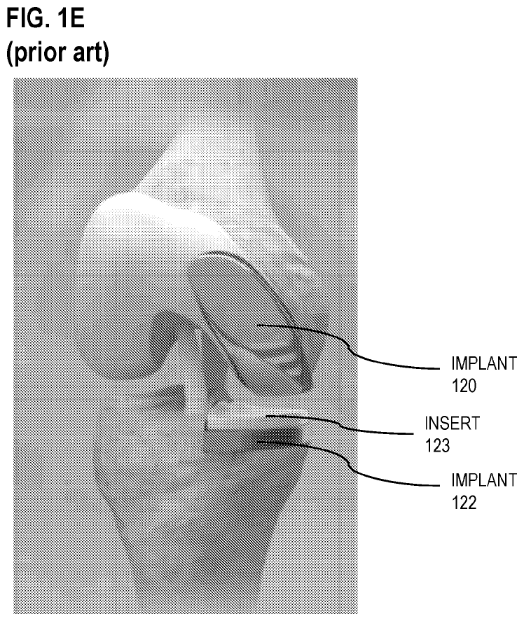

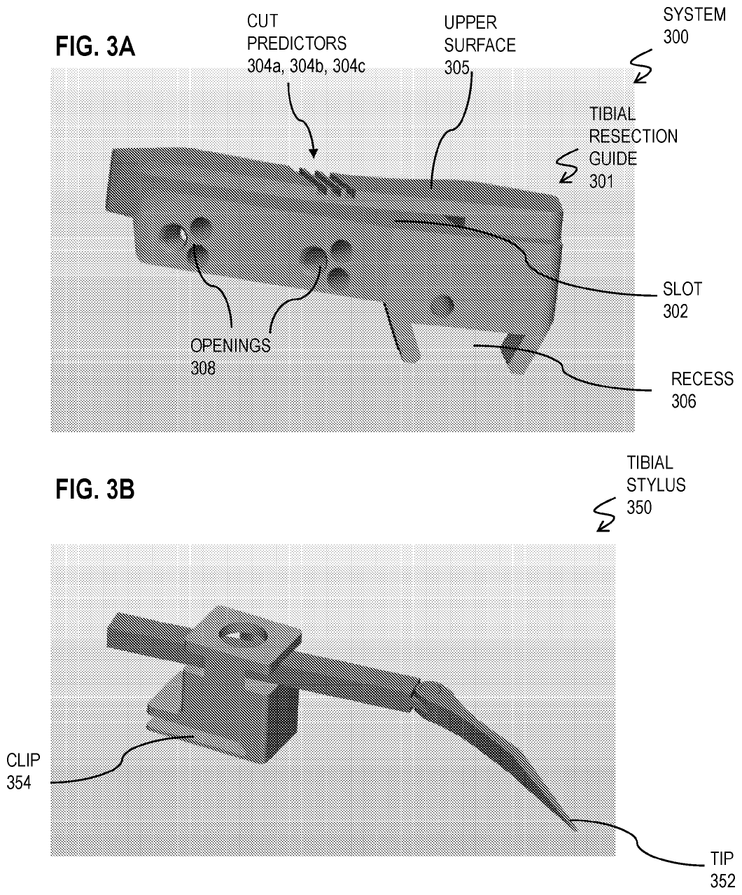

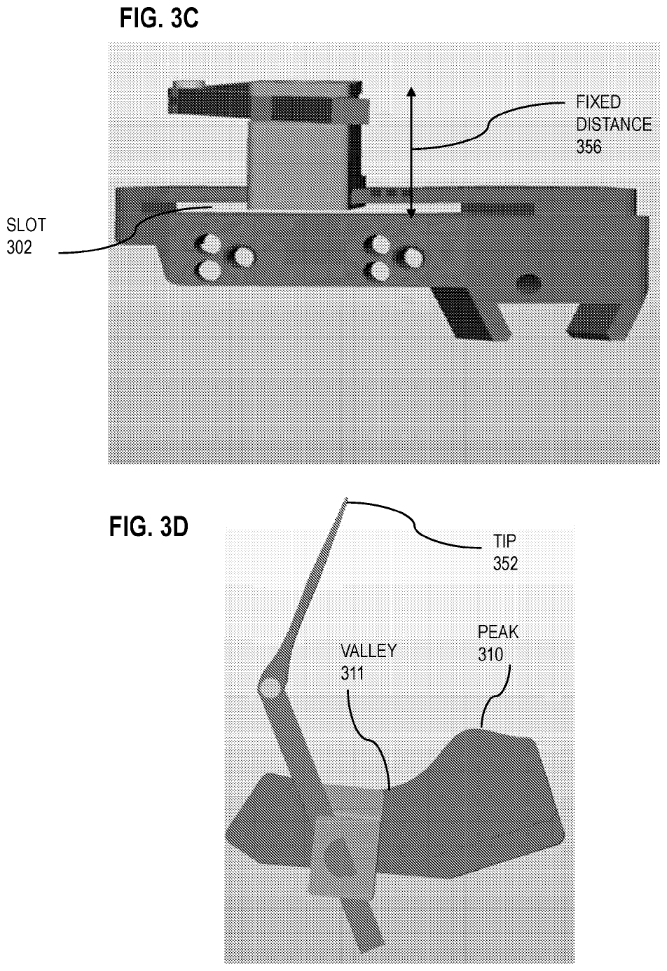

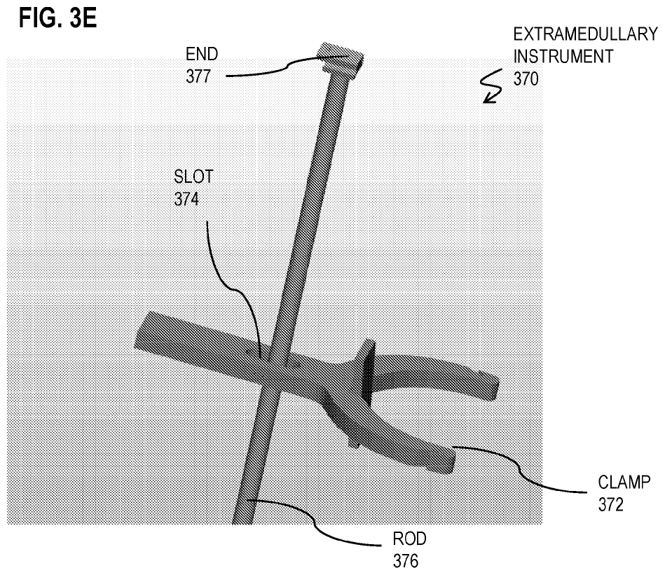

A step of cutting a portion of the tibia 104 will now be discussed, in order to form one or more cut surfaces in the tibia 104 that are used in performing the unicompartmental knee arthroplasty. In step 202, a portion of the tibial plateau 109 (FIG. 1B) is cut with one or more first cutting devices to form one or more cut surfaces in the tibial plateau 109. FIGS. 3A through 3E are images that illustrate an example of first cutting devices 300, 350, 370 of a system 300 that are used to perform the cutting step 202 of the method 200 to form cut surfaces in the tibial plateau 109, according to one embodiment.

In an embodiment, as shown in FIG. 3A, the first cutting device includes a tibial resection guide 301 configured to be mounted to the tibia 104 and defining a slot 302 to slidably receive a cutting instrument (e.g. knife or saw) to form one of the cut surfaces. In an example embodiment, the slot 302 has an adjusted thickness to accommodate thicker saw blades. The tibial resection guide 301 defines a plurality of cut predictors 304a, 304b, 304c on an upper surface 305 of the tibial resection guide 301. In one embodiment, the cut predictors 304a, 304b, 304c are utilized in mounting the tibial resection guide 301 to the tibia 104. In an example embodiment, the cut predictors 304a, 304b, 304c are utilized to ensure that when the tibial resection guide 301 is mounted to the tibia 104 and the cutting instrument is moved within the slot 302 to cut the tibial plateau 109, the cutting instrument docs not come within a threshold distance of certain anatomical features of the tibia (e.g. intercondylar eminence). In still another example embodiment, the cut predictors 304a, 304b, 304c are engraved on the upper surface 305 of the tibial resection guide 301 for referencing a general direction of the medial intercondylar eminence of the tibia (MICET).

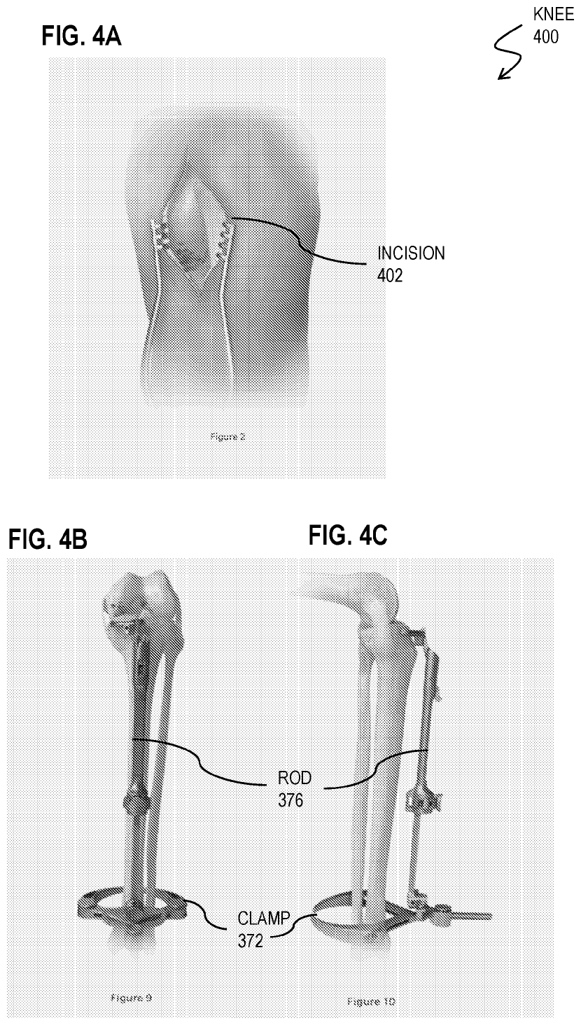

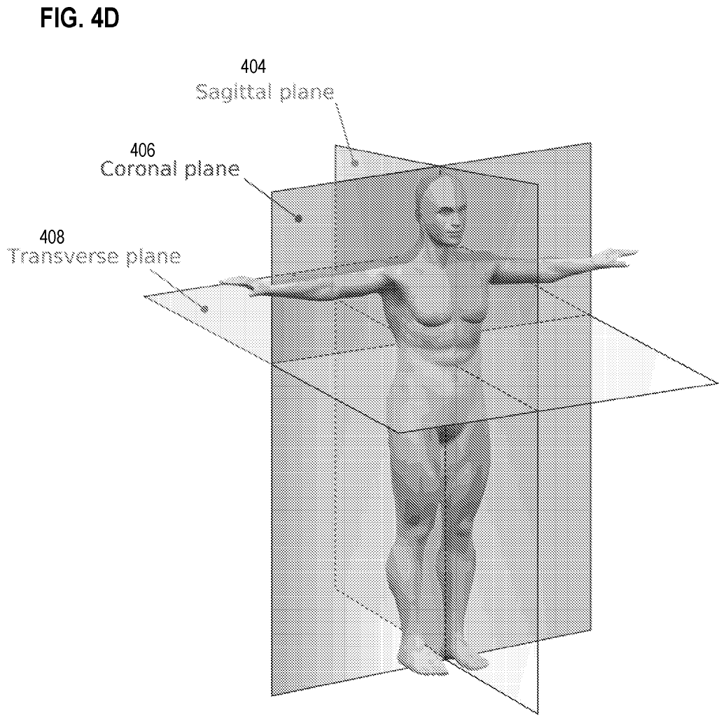

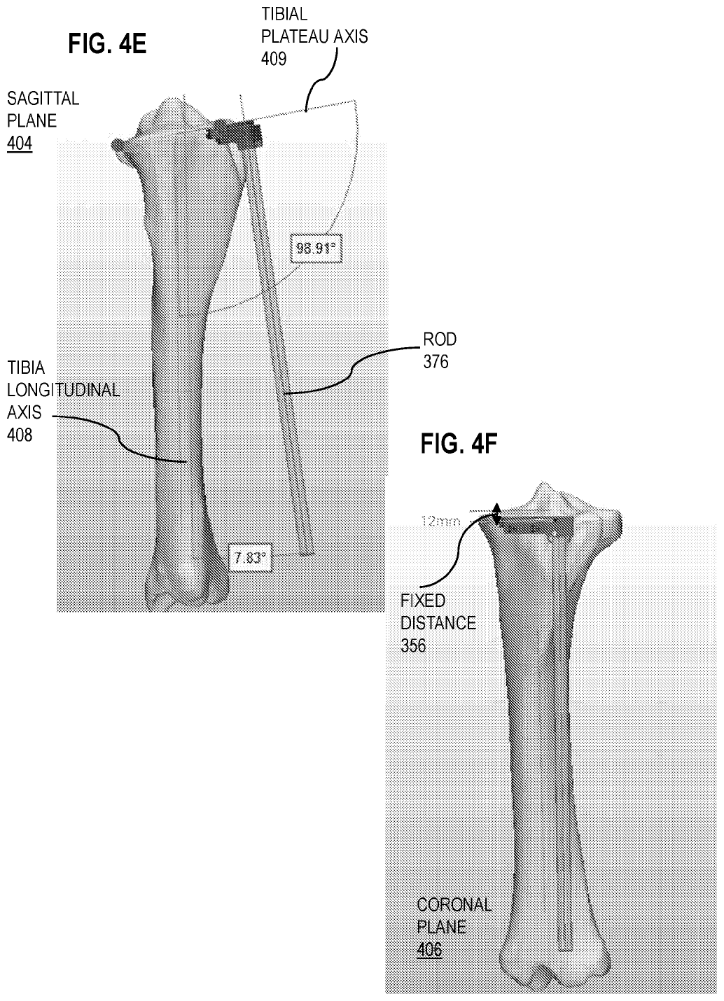

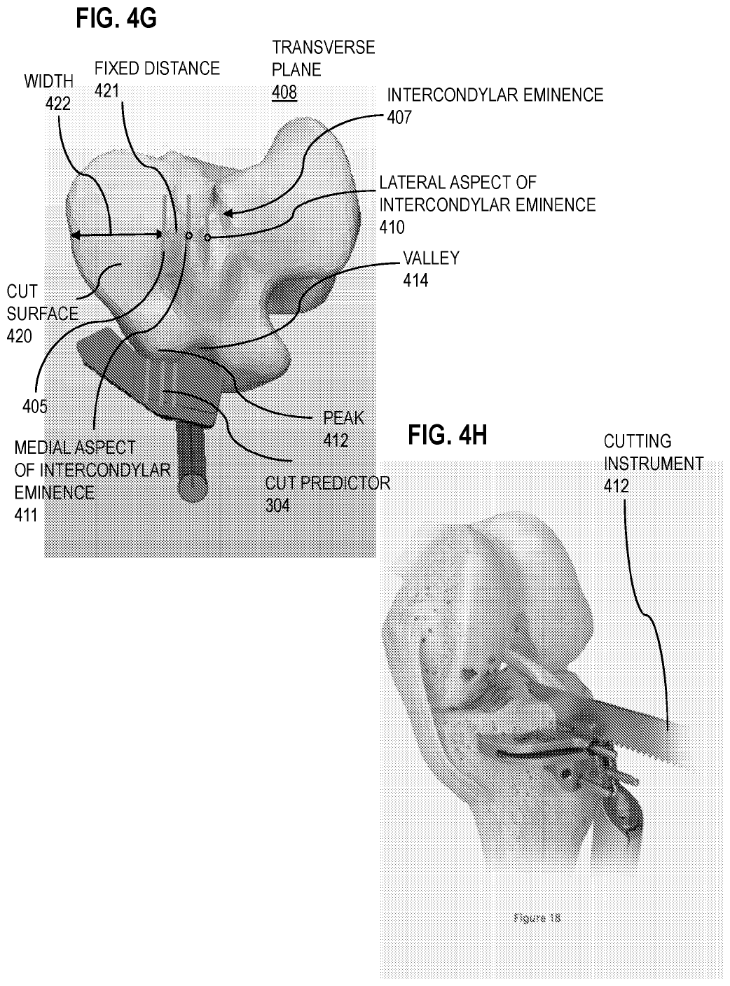

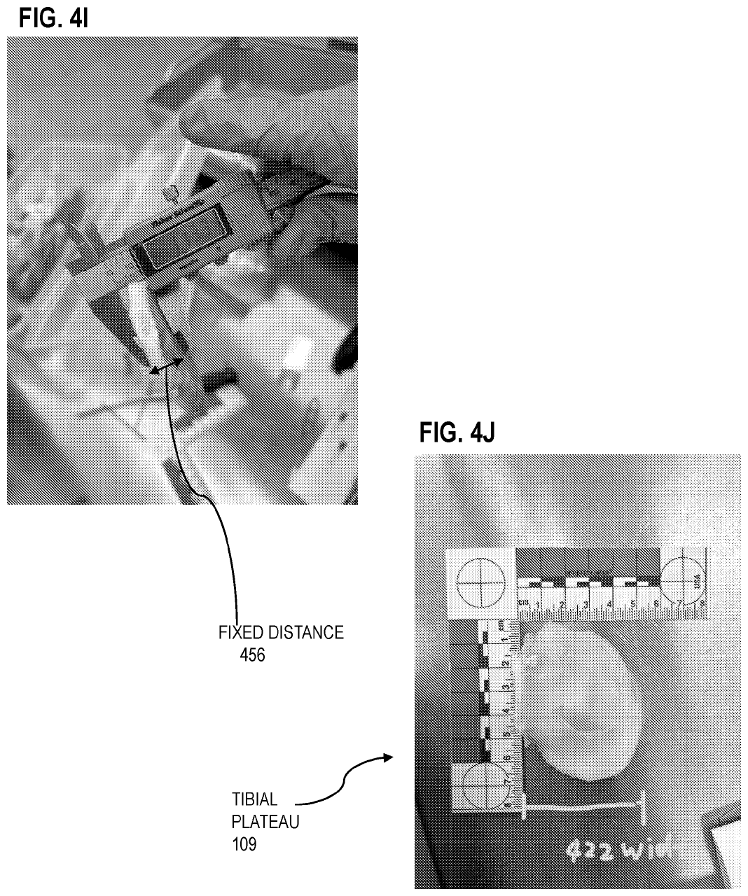

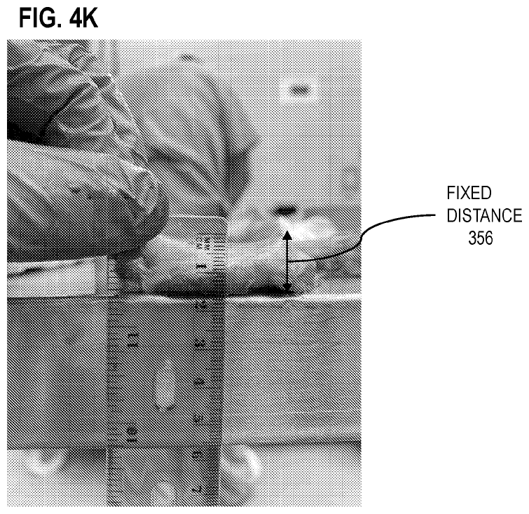

In an embodiment, the tibial resection guide 301 includes features to mount the tibial resection guide 301 to the tibia 104. In one embodiment, the tibial resection guide 301 defines one or more openings 308, to securely mount the tibial resection guide 301 to the tibia 104 (e.g. by passing one or more fasteners through the openings 308 and into the tibia 104). In one embodiment, the openings 308 are three horizontally paired openings with a certain height difference (e.g. within about±2 mm). However, in other embodiments other means appreciated by one of ordinary skill in the art can be used to securely mount the tibial resection guide 301 the tibia 104. Additionally, as shown in FIG. 3D, a side of the tibial resection guide 301 that is configured to engage the tibial plateau 109 is shaped with a curvature including a peak 310 and valley 311 that is shaped to engage corresponding curvature in the tibial plateau 109. Tibial tuberosity (TT) is split up in the horse into a lateral TT and a medial TT. As shown in FIG. 4G, the shape of the tibial plateau 109 includes the peak 412 that is the Medial TT and the valley 414 that is the intertubecular groove. As further shown in FIG. 4G, the peak 310 and valley 311 of the tibial resection guide 301 are shaped to match the respective valley 414 and peak 412 in a side of the tibial plateau 109.

Additionally, in an embodiment the first cutting device includes a tibial stylus 350 (FIG. 3B) configured to be mounted to the tibial resection guide 301. In one embodiment, the tibia stylus 350 includes a tip 352 configured to define a predefined distance 356 (FIG. 3C) between the tip 352 and the slot 302 when the tibial stylus 350 is mounted to the tibial resection guide 301. The predefined distance 356 is utilized during the cutting step 202 to restrict a thickness of the tibia plateau 109 that is cut (e.g. to be no more than the predefined distance 356 in one dimension).

Additionally, in an embodiment the system includes an extramedullary instrument 370 (FIG. 3E) that is utilized in mounting the tibial resection guide 301 to the tibia 104. In an example embodiment, the extramedullary instrument 370 is utilized to adjust an alignment of the tibial resection guide 301 (e.g. such that it is aligned with a surface of the tibial plateau 109). In one embodiment, the extramedullary instrument 370 includes a clamp 372 defining a slot 374, where the clamp 372 is configured to be secured to a lower leg of the mammal below the knee. In this embodiment, the extramedullary instrument 370 also includes a rod 376 configured to pass through the slot 374. The rod 376 has a first end and a second end 377 opposite the first end. The second end 377 is received within a recess 306 (FIG. 3A) defined by the tibial resection guide 301. In an example embodiment, the extramedullary instrument 370 provides different (e.g. 5) alignment options with 1 degree increment ranging from about 6 degrees to about 10 degrees allowing for the tibial resection to achieve 7 degree to 9 degree of caudal tibial slope angle.

FIGS. 4A through 4K are images that illustrate an example of the first cutting devices of FIGS. 3A through 3E being used to perform the cutting step 202 to form the cut surfaces in the tibial plateau 109, according to one embodiment. As previously discussed, in step 202 the first cutting devices 301, 350, 370 are utilized to cut a portion of a plateau 109 of the tibia to form one or more cut surfaces in the tibial plateau 109. As shown in FIG. 4A, in an initial step an incision 402 is formed in the knee 400, to provide access to the knee joint and the tibial plateau 109. FIG. 4D depicts three planes that are utilized in the discussion of the method 200. A sagittal plane 404 is defined as the plane that bifurcates the subject into left/right; a coronal plane 406 is defined as the plane that bifurcates the subject into front/back and a transverse plane 408 that is perpendicular to both the sagittal plane 404 and coronal plane 406. Although a human subject is shown in FIG. 4D, the same planes are defined with respect to non-human subjects (e.g. horse).

In these embodiments, the tibial resection guide 301 is mounted to the tibia 104 (e.g. to the tibial plateau 109). In some embodiments, the extramedullary instrument 370 is utilized in mounting the tibial resection guide 301 to the tibial plateau 109. FIGS. 4B through 4F depict the mounting of the tibial resection guide 301 to the tibial plateau 109 with the extramedullary instrument 370. In an embodiment, step 202 includes securing the clamp 372 of the extramedullary instrument 370 to a lower leg of the mammal below the knee (FIGS. 4B and 4C). In these embodiments, the extramedullary instrument 370 is used to vary an alignment of the tibial resection guide 301 within the sagittal plane 404. In an example embodiment, in step 202 the extramedullary instrument is used to align the tibial resection guide 301 with an axis 409 (FIG. 4E) defined by a surface of the tibial plateau 109 in the sagittal plane 404. The second end 377 of the rod 376 is secured within the recess 306 of the tibial resection guide 301 and thus varying an alignment of the rod 376 in the sagittal plane 404 also varies an alignment of the tibial resection guide 301 in the sagittal plane 404. In an example embodiment, to adjust the alignment of the tibial resection guide 301 in the sagittal plane 404, a position of the rod 376 within the slot 374 of the clamp 372 is varied, which varies an alignment of the rod 376 (and hence the tibial resection guide 301), as shown in FIG. 4E. In some embodiments, the angle of the tibial resection guide 301 relative to a longitudinal axis 408 of the tibia 104 is varied until the tibial resection guide 301 is aligned with the axis 409 defined by the surface of the tibial plateau 109. In an example embodiment, the angle is in a range between about 90 degrees and about 120 degrees and/or in a range between about 80 degrees and about 130 degrees.

In an embodiment, the tibial stylus 350 is mounted to the tibial resection guide 301. In an embodiment, a clip 354 (FIG. 3B) of the tibial stylus 350 is mounted to the upper surface 305 of the tibial resection guide 301. As previously discussed and as shown in FIG. 4F, the tibial stylus 350 includes the tip 352 to define the predefined distance 356 in a vertical direction in the coronal plane 406 between the tip 352 and the slot 302 of the tibial resection guide 301. In one embodiment, in step 202 the tip 352 of the tibial stylus 350 is positioned adjacent the plateau 109 of the tibia such that the predefined distance 356 in the vertical direction in the coronal plane 406 is defined between the plateau 109 of the tibia and the slot 302 of the tibial resection guide 301. The inventors of the present invention recognized that these structural features of the tibial resection guide 301 and tibial stylus 350 advantageously ensures that a dimension of the tibial plateau 109 removed in the vertical direction within the coronal plane 406 is no greater than a threshold amount (e.g. the predefined distance 356).

In these embodiments, the cutting step 202 includes moving the cutting instrument along the slot 302 to remove a thickness of the tibia 104 in the vertical direction in the coronal plane 406 that does not exceed the predefined distance 356. In an example embodiment, FIG. 4K depicts an example of the removed thickness of the tibial plateau 109 in the vertical direction in the coronal plane 406 (e.g. about equal to the predefined distance 356). In another example, FIG. 4J depicts an example of a spacing between the cut surface in the tibial plateau 109 and the medial condyle as a result of removing the thickness of the tibial plateau 109 shown in FIG. 4K (e.g. that is about equal to the predefined distance 356). In an example embodiment, the predefined distance 356 is about 12 millimeters (mm)±2 mm. The inventors determined that a minimum thickness for the tibial baseplate to be about 4 mm in human UKA. The inventors further determined that due to greater forces in horse stifles, an estimate of a minimum thickness for the tibial baseplate for horses should be greater than 4 mm (e.g. about 6 mm). The inventors noted that the tibial insert thickness in human UKA ranged from about 4 mm to about 10 mm. Hence, in one embodiment, the thickness estimate for the tibial insert for UKA with horses was set to be about 6 mm and above. Consequently, in these embodiments, the value of the predefined distance 356 was set based on a sum of the tibial baseplate thickness and tibial insert thickness for UKA with horses.

In an embodiment, step 202 includes mounting the tibial resection guide 301 to the tibia 104. As shown in FIG. 4G, the tibia 104 includes an intercondylar eminence 407 that has a lateral aspect 410 and a medial aspect 411. In one embodiment, in step 202 mounting the tibial resection guide 301 to the tibia 104 includes aligning one of the cut predictors 304a, 304b, 304c on the upper surface 305 of the tibial resection guide 301 with a region 405 of the tibia 104. In one embodiment, the region 405 is spaced apart from the medial aspect 411 of the intercondylar eminence 407 by no less than a fixed distance 421 (e.g. about 12 mm). The inventors of the present invention recognized that this ensures that a most lateral portion of the slot 302 is no closer than the threshold distance 421 (e.g. about 12 mm) from the medial aspect 411 of the intercondylar eminence 407 in the traverse plane 408. In an example embodiment, the threshold distance 421 is scaled with a dimension to avoid cutting into the intercondylar eminence 407 and damaging the attachment of the cranial cruciate ligament to the tibia 104. In an example embodiment, the threshold distance 421 of about 12 mm or in a range from about 10 mm to about 14 mm is based on measurements performed on a series of equine tibias, and dimensional values were judged to be a reasonable figure for a safe distance for most sizes of tibiae 104. In some embodiments, the cut predictors 304a, 304b, 304c do not play a primary role in the attachment of the tibial resection guide 301. In these embodiments, the primary factors are the transverse plane alignment, facilitated by the peak 310/valley 311 features as described, and the sagittal plane 404 alignment, including the correct height and the correct angle for the resected tibial plateau slope. Once properly attached, the cut predictors 304a, 304b, 304c are used as visual reference guides for the surgeon to choose how to perform the vertical tibial cut. The inventors of the present invention recognized that this advantageously ensures that the cut surface 420 formed by the cutting step 202 is not closer than the threshold distance 421 from the medial aspect 411 of the intercondylar eminence 407 of the tibia 104 in the traverse plane 408.

In an embodiment, FIG. 4G depicts the cut surface 420 that has a width 422 in the traverse plane 408 which is the result of step 202 where the cutting instrument 412 is moved through the slot 302 after the tibial resection guide 301 is properly mounted to the tibial plateau 109. FIG. 4I depicts an example embodiment of the cut portion of the tibial plateau 109 in step 202 with the fixed distance 456. In an example embodiment, the fixed distance 456 is about 11 mm or in a range from about 8 mm to about 14 mm. FIG. 4J depicts an example embodiment of the cut portion of the tibial plateau 109 in step 202 with the width 422. In an example embodiment, the width 422 is in a range from about 32 mm to about 40 mm or in a range from about 25 mm to about 50 mm. As shown in FIG. 4G when the tibial resection guide 301 is mounted to the tibial plateau 109, the surface of the tibial resection guide has a curvature that is shaped (e.g. see peak 310 and valley 311 in FIG. 3D) so to compliment the curvature of the side of the tibial plateau 109. In an example embodiment, as shown in FIG. 4G, the peak 412 and valley 414 along the side of the tibial plateau 109 fit within the respective valley 311 and peak 310 of the side of the tibial resection guide 301.

In an embodiment, in step 202 in addition to passing the cutting instrument 412 along the slot 302 to form the cut surface 420 in the tibial plateau 109, the cutting instrument 412 is also used to form a second cut surface (FIG. 4H) that is oriented about orthogonal to the cut surface 420. In these embodiments, after the cutting instrument 412 forms the cut surface 420 and the second cut surface, a portion of the tibial plateau 109 is removed, leaving a cavity to mount an implant. FIGS. 4I through 4K depict the removed portion of the tibial plateau in step 202. In some embodiments, the cut predictors 304a, 304b, 304c are utilized by the surgeon to provide visual cues to align the cutting instrument 412 in performing the cut to form the second cut surface. In other embodiments, the tibial resection guide 301 may define a vertical cutting slot to pass the cutting instrument 412 in performing the cut to form the second cut surface.

### 1.2 Cutting the Femur

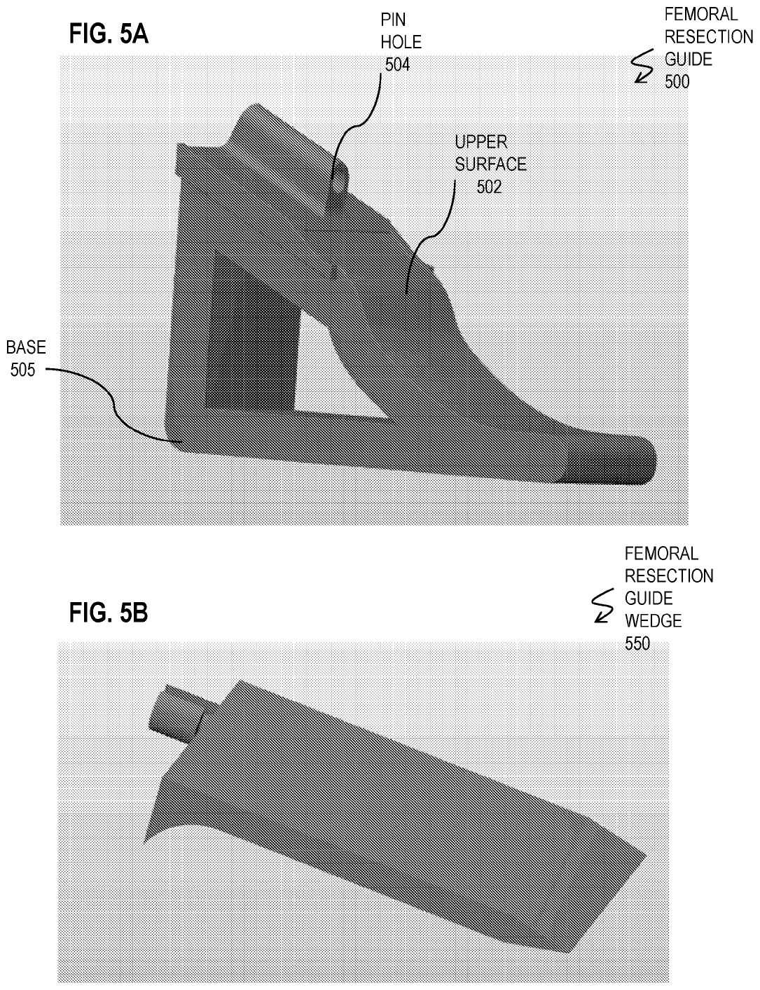

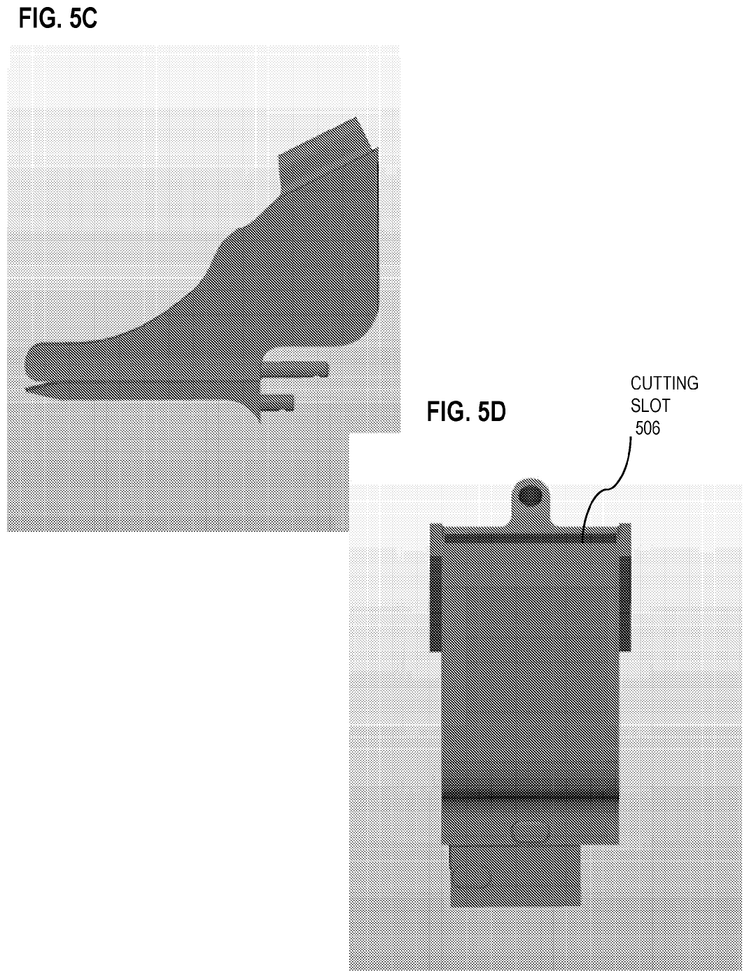

A step of cutting a portion of the femur 102 will now be discussed, in order to form one or more cut surfaces in the femur 102 that are used in performing the unicompartmental knee arthroplasty. In step 204, a portion of a medial condyle 108 of the femur 102 is cut with a second cutting device to form cut surfaces in the medial condyle 108. FIGS. 5A through 5D are images that illustrate an example of a second cutting device 500, 550 of the system 300 that are used to perform the cutting step 204, to form a cut surface in the medial condyle 108, according to one embodiment.

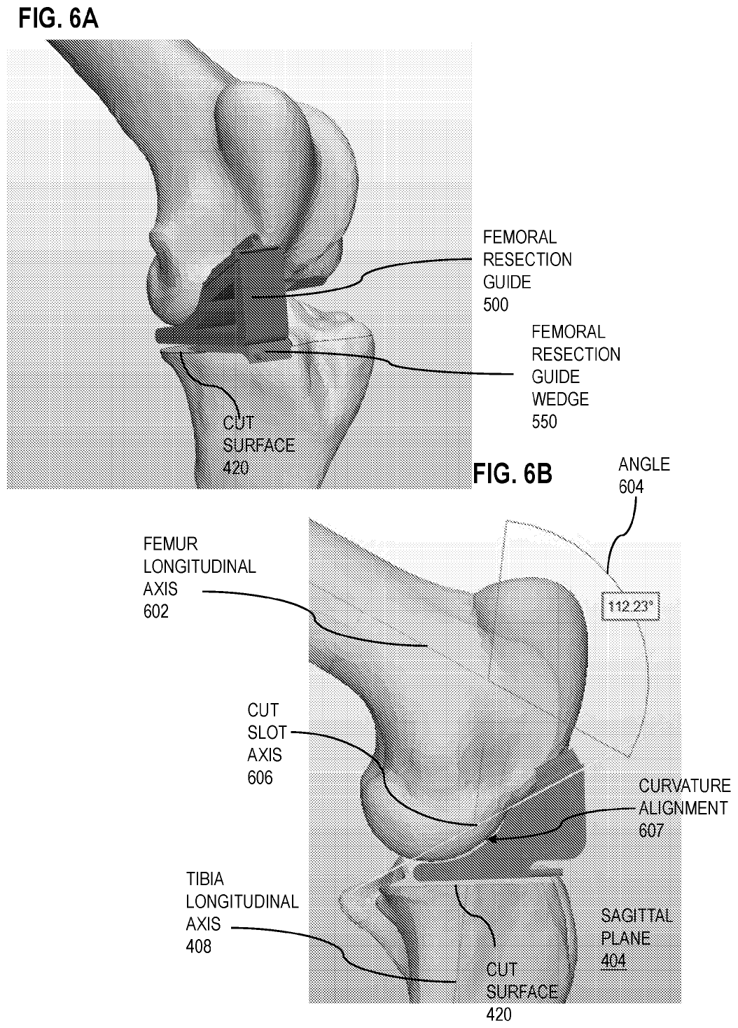

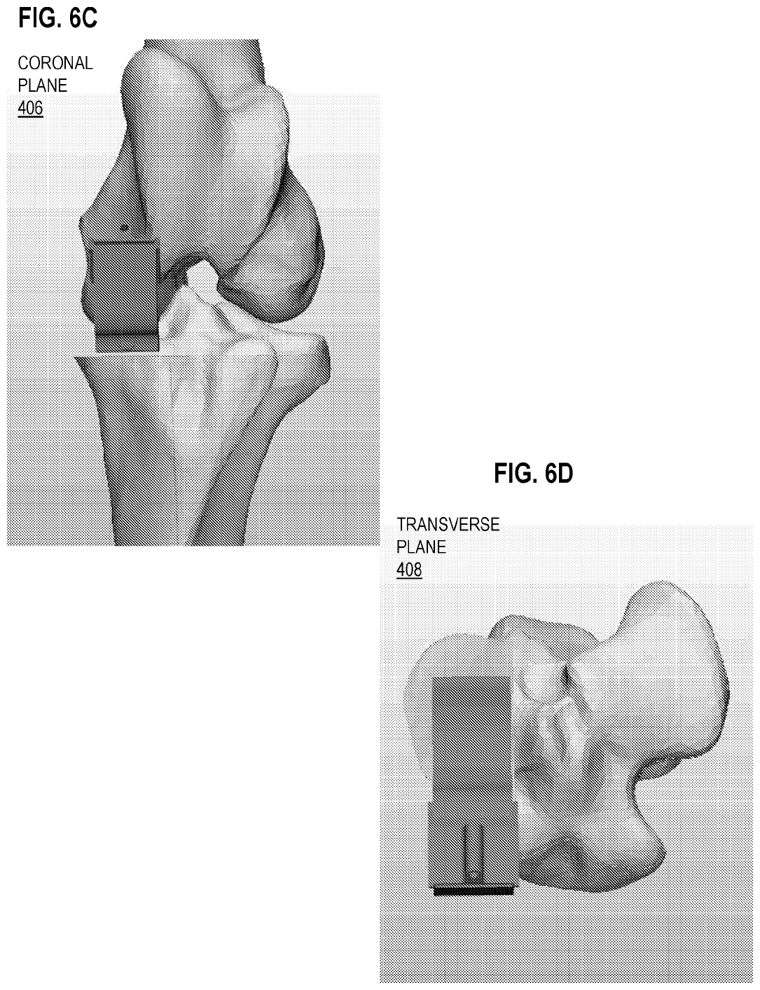

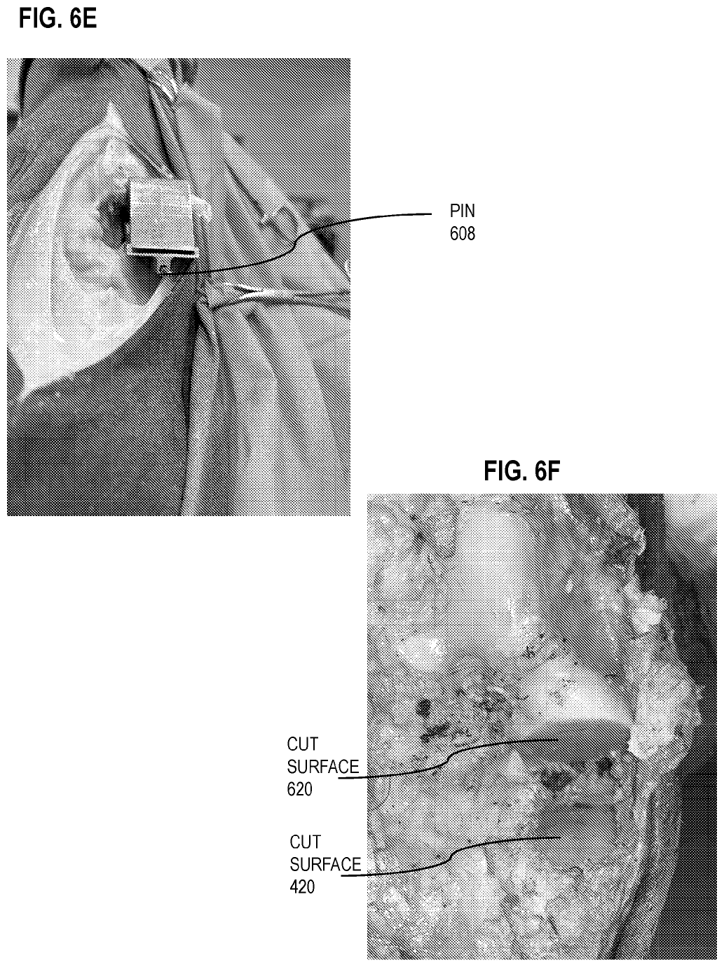

In an embodiment, the second cutting device includes a femoral resection guide 500 configured to be mounted to the medial condyle 108 to form one of the second cut surfaces of the medial condyle 108. In one embodiment, as shown in FIG. 5A the femoral resection guide 500 includes an upper surface 502 that is shaped to match a shape of an outer surface of the medial condyle. The inventors of the present invention recognized that this step is distinct from a conventional method involving human UKA. Quadrupeds generally do not fully extend their knees, and so bringing the knee into full extension (as is commonly done to reference the sagittal alignment in human UKA) is not done in the embodiments herein. Instead, the inventors of the present invention developed this specific device (femoral resection guide 500) to allow accurate cuts of the flexed femur, aligning with the cartilage line that delineates the margin of the femora-tibial joint and the patella-femoral joint. This feature, and the peak/valley of the tibial resection guide 301 are two distinguishing features of the bone preparation process that differ from the conventional human approach. See the cut slot axis 606 in FIG. 6B which depicts how the femoral resection guide 500 is specifically shaped and configured to allow this accurate cut.

Additionally, in these embodiments, the femoral resection guide 500 defines a pin hole 504 configured to receive a pin to be inserted in the fossa which is between the medial condyle 108 and the medial trochlear ridge to secure the femoral resection guide 500 to the medial condyle 108. Additionally, in an embodiment, the femoral resection guide 500 defines a cutting slot 506 (FIG. 5D) to slidably receive a cutting instrument to form the one of the second cut surfaces.

In an embodiment, the second cutting device also includes a femoral resection guide wedge 550 (FIG. 5B) configured to be positioned between a base 505 of the femoral resection guide 500 and the first cut surface 420 in the tibial plateau 109 to prevent the medial compartment 114 of the knee from collapsing during step 204. For purposes of this description, preventing “collapse” of the medial compartment 114 means maintaining appropriate bone alignment and to achieve cuts appropriate for the implant construct thickness. Although a single femoral resection guide wedge 550 is depicted in FIG. 5B, the embodiments of the present invention include a plurality of femoral resection guide wedges having different dimensions (e.g. different thickness in a vertical direction along a longitudinal axis of the tibia) so that one of the femoral resection guide wedges is selected based on the particular dimensions of the knee joint where the method 200 is performed. In an example embodiment, the different thicknesses of the femoral resection guide wedges are about 4 mm, about 5 mm and about 6 mm.

FIGS. 6A through 6F are images that illustrate an example of the second cutting device 500, 550 of FIGS. 5A through 5D being used to perform the cutting step 204 to form the cut surface in the medial condyle 108, according to one embodiment. In an embodiment, in step 204 the femoral resection guide 500 is mounted to the medial condyle 108 to form one of the second cut surfaces of the medial condylc. In these embodiments, the upper surface 502 of the femoral resection guide 500 is positioned to contact a surface of the medial condyle 108. In an example embodiment, the curvature of the upper surface 502 is selected to match a curvature of the medial condyle 108. As shown in FIG. 6B, a curvature alignment 607 is depicted where the cartilage of the medial condyle 108 has a curvature that aligns with the curvature of the upper surface 502. FIGS. 6A and 6B depict the femoral resection guide 500 positioned with the base 505 on the cut surface 420 in the tibial plateau 109 (from step 202). The upper surface 502 has a curvature that matches a curvature of the medial condyle 108. Additionally, in step 204 the femoral resection guide wedge 550 is positioned between the base 505 of the femoral resection guide 500 and the cut surface 420. In these embodiments, one of a plurality of femoral resection guide wedges 550 is selected based on a thickness of the femoral sector wedge 550 fits between the base 505 and the cut surface 420 in order to prevent collapse of the medial compartment 114 of the knee.

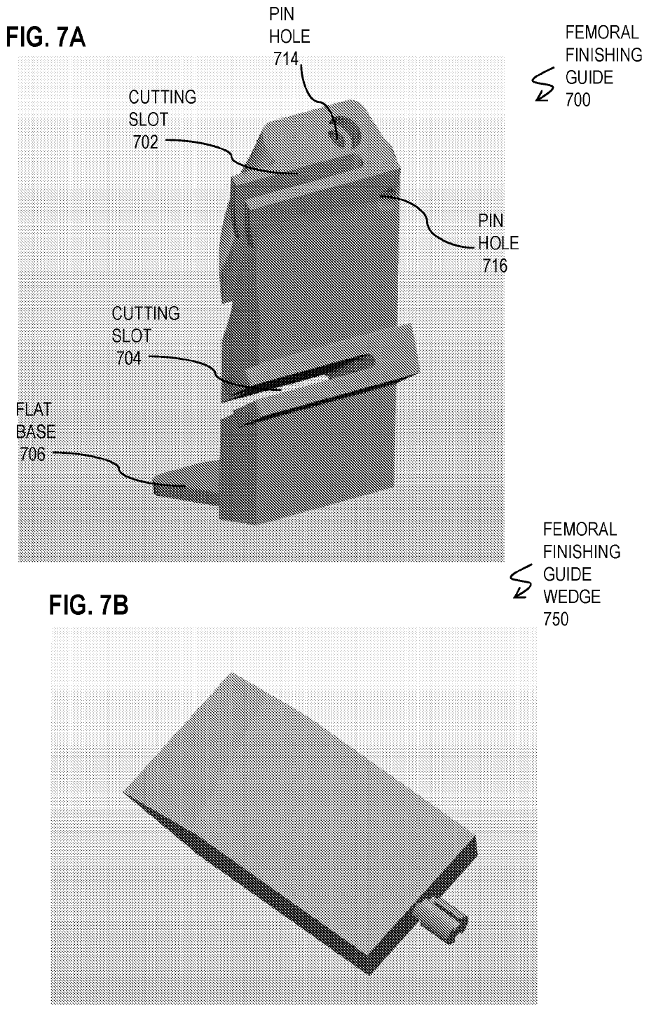

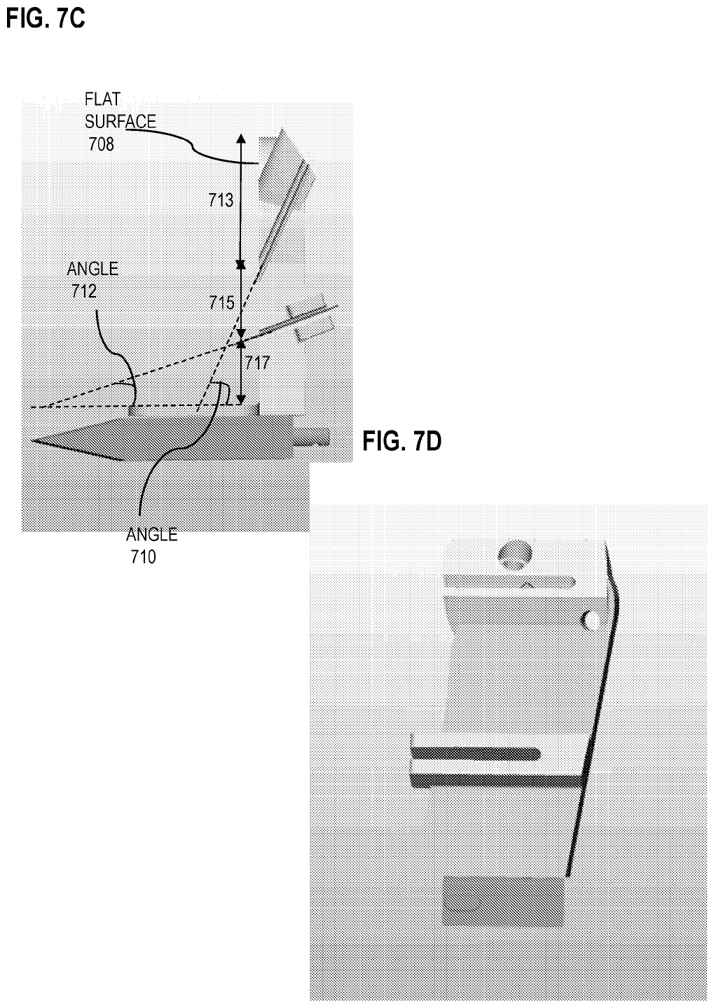

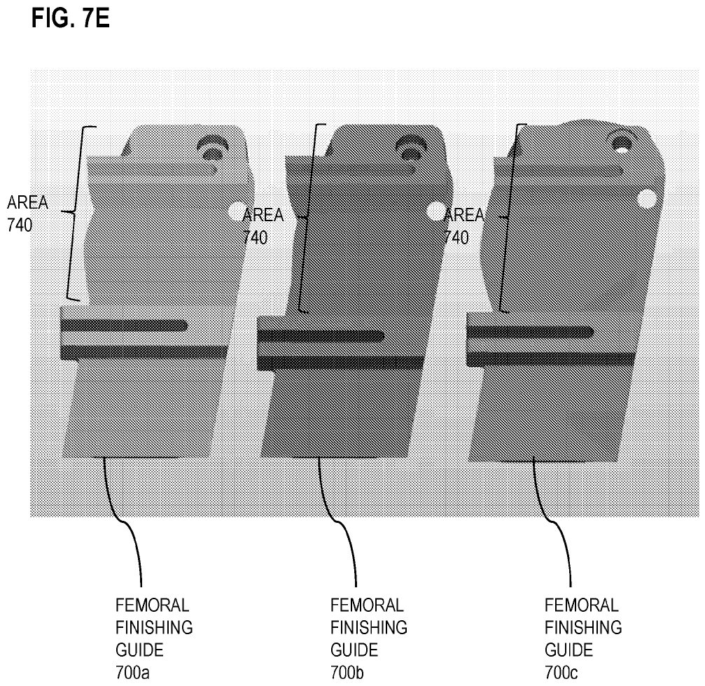

In step 204, after positioning the femoral resection guide 500 as shown in FIGS. 6A and 6B, a pin 608 (FIG. 6E) is passed through the pin hole 504 and into a most proximal edge of the medial condyle 108 to secure the femoral resection guide 500 to the medial condyle 108. Although “pin and “pin hole” are used herein, for purposes of this description “pin” and “pin hole” respectively include screw and screw holes. In one example embodiment, the pin 608 is a self-tapping screw (e.g. 3.5 mm) that is used in step 204. In this example embodiment, when using the screws, a screw hole (e.g. 2.5 mm) is drilled through the bone first. Then, in step 204 the screw is applied through the hole 504 in the femoral resection guide 500 and the screw hole drilled in the bone, which tightens the femoral resection guide 500 down to the bone. As shown in FIG. 6B upon positioning the femoral resection guide 500 and passing the pin through the pin hole 504, a cut slot axis 606 defined by the cutting slot 506 of the femoral resection guide 500 is aligned with a segment of the medial condyle 108. Thus, in these embodiments, upon passing the cutting instrument along the cutting slot 506, a second cut surface 620 (FIG. 6F) is formed in the medial condyle 108. As shown in FIG. 6B, the cutting step 204 is performed when the longitudinal axis 408 of the tibia 104 and the longitudinal axis 602 of the femur 102 have a particular angle 604 between them. In one example embodiment, the angle 604 is in a range from about 105 degrees to about 115 degrees and/or from about 90 degrees to about 130 degrees. The inventors of the present invention recognized that this angle 604 is effective in performing the cutting step 204, particularly in non-human mammals (e.g. horses) who have distinct anatomical differences with human mammals. As previously discussed, in some embodiments the angle 604 is the result of flexing the knee so that the T-F/P-F cartilage junction aligns with the femoral resection guide 500. In one example embodiment, the inventors of the present invention recognized that with the horse UKA the most difficult cut to perform is the cut that corresponds to angle 710 (FIG. 7C). The inventors of the present invention further recognized that this cut becomes easier, the more acute angle 604 is. However, the inventors of the present invention recognized that the angle 604 is limited because if the angle 604 is too acute then the femoropatellar joint would be aligned to be cut, which is not desired.

After forming the cut surface 620 in the medial condyle 108 with the femoral resection guide 500, step 204 includes using another second cutting device to form additional cut surfaces in the medial condyle 108 using the cut surface 620. FIGS. 7A through 7E are images that illustrate an example of a second cutting device of a system 300 that is also used to perform the cutting step 204 to form additional cut surfaces in the medial condyle, according to one embodiment. In an embodiment, the second cutting device is a femoral finishing guide 700 configured to be mounted to the second cut surface 620 formed by the femoral resection guide 500. The femoral finishing guide 700 is configured to form multiple cut surfaces in the medial condyle 108. As shown in FIG. 7A, the femoral finishing guide 700 defines a pair of slots 702, 702 that are oriented at different angles and are each configured to receive the cutting instrument 412 to form the plurality of the second cut surfaces in the medial condyle 108.

In an embodiment, as shown in FIG. 7C a first slot 702 of the pair of slots 702, 704 forms a first angle 710 relative to a flat base 706 of the femoral finishing guide 700 and a second slot 704 of the pair of slots 702, 704 forms a second angle 712 relative to the flat base 706 that is smaller than the first angle 710. In an example embodiment, the first angle 710 is about 65 degrees or in a range from about 63 degrees to about 67 degrees or from about 60 degrees to about 70 degrees and the second angle 712 is about 20 degrees or in a range from about 18 degrees to about 22 degrees or from about 15 degrees to about 25 degrees. In these example embodiments, the value of these angles 710, 712 is selected in order that the cut surfaces formed in the medial condyle 108 are at respective angles to accommodate mounting an implant to the cut surfaces. In other embodiments the values of the angles 710, 712 are the same for different femoral finishing guides 700 but the value of the lengths 713, 715, 717 can be sized with different dimensions (e.g. 5 different sized femoral finishing guides) to accommodate different sized knee joints. In some embodiments, the inventors of the present invention recognized that the angles 710, 712 result from the design of the femoral implant. In these embodiments, the goal is to resect as little bone as possible with no more than three cut surfaces, while maintaining a minimum thickness of the implant at the chamfer (thinnest point). In these embodiments, the femoral implant design was based on so-called statistical shape models of the horse femur that represent the most likely bone shape in S/M/L sizes. In an example embodiment, statistical shape model matching for the medium and large shaped implants is mean +/−1 SD. The femoral finishing guide was design to cover the required sagittal arc of bone surface, and then to have no more than three inner facets with an overall minimum thickness.

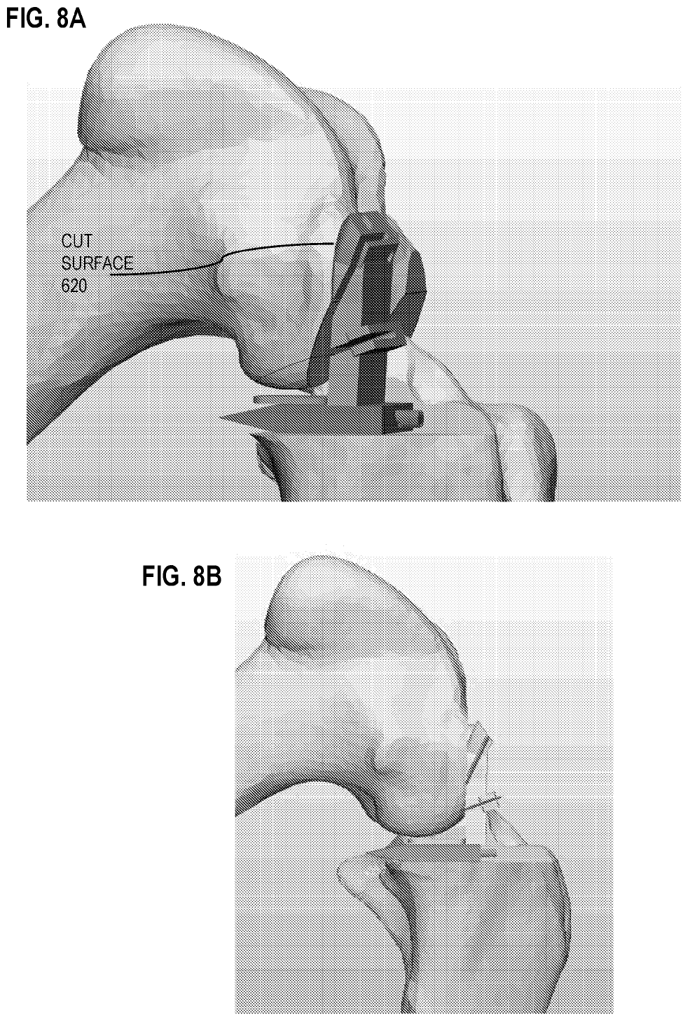

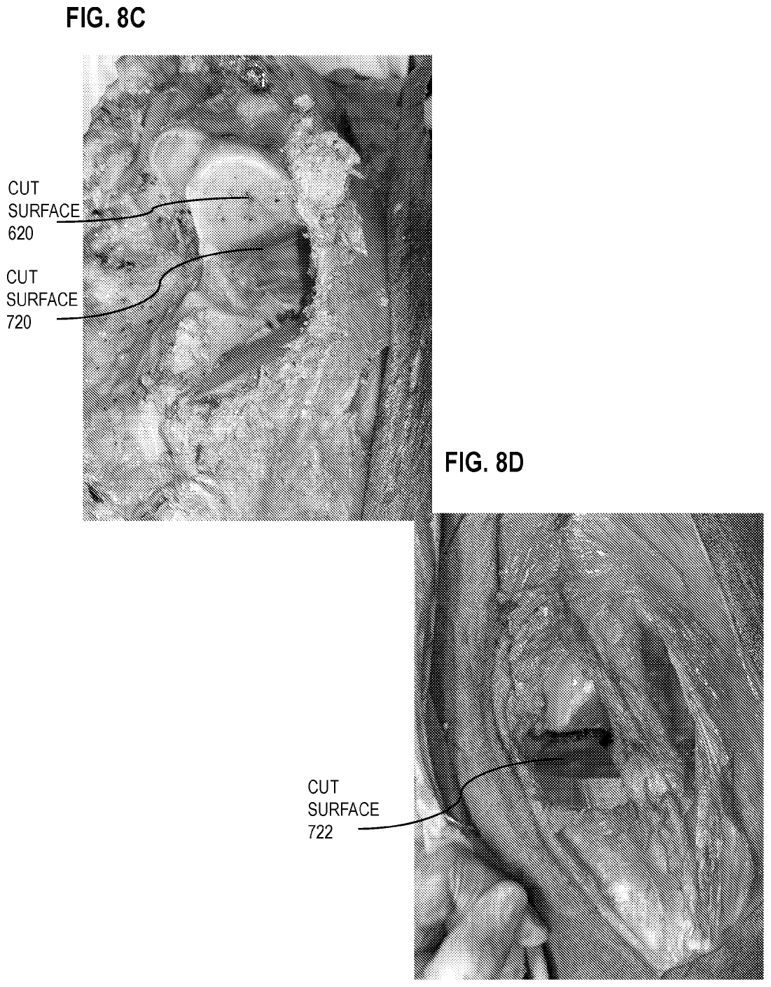

In an embodiment, as shown in FIG. 7C the femoral finishing guide 700 includes a flat surface 708 adjacent to the pair of slots 702, 704 such that upon positioning the flat surface 708 against the second cut surface 620 (FIG. 8A) formed by the femoral resection guide 500, the pair of slots 702, 704 are aligned with the medial condyle 108 to form the plurality of second cut surfaces with the cutting instrument 412. Additionally, as shown in FIG. 7A, the femoral finishing guide 700 defines a pair of converging pin holes 714, 716 that are configured to receive respective pins (not shown) to secure the femoral finishing guide 700 to the second cut surface 620 formed by the femoral resection guide 500. The femoral finishing guide 700 includes the flat base 706 that is connected to the flat surface 708.

In an embodiment, as shown in FIG. 7B the system 300 further includes a femoral finishing guide wedge 750 configured to be positioned between the flat base 706 of the femoral finishing guide 700 and the first cut surface 420 in the tibial plateau 109 (FIG. 8A) to prevent a medial compartment 114 of the knee from collapsing during step 204.

FIGS. 8A through 8D are images that illustrate an example of the femoral finishing guide 700 of FIGS. 7A through 7E being used to perform the cutting step 204 to form the additional cut surfaces in the medial condyle, according to one embodiment. In an embodiment, in step 204 the femoral finishing guide 700 is mounted to the second cut surface 620 formed by the femoral resection guide 500 to form a plurality of the second cut surfaces. In some embodiments, this step involves selecting the femoral finishing guide 700 among a plurality of femoral finishing guides 700a, 700b, 700c (FIG. 7E) of different dimensions. In an example embodiment, the femoral finishing guide 700 is selected based on the dimensions of the surfaces of the knee joint (e.g. dimensions of the cut surface 420, dimensions of the cut surface 620), which may depend on an age of the mammal (e.g. age of the horse). In this example embodiment, to aid with size selection, the plurality of femoral finishing guides 700a, 700b, 700c were formed so the flat surface 708 aligns with the medial extent of the cut surface 620 (see the brackets depicted in FIG. 7E which indicate the area 740 of the femoral finishing guides 700a, 700b, 700c that align with the medial limit of the cut surface 620).

In an embodiment, to mount the femoral finishing guide 700 to the second cut surface 620, respective pins (not shown) are passed through the pair of converging pin holes 714, 716 defined by the femoral finishing guide 700 and into the second cut surface 620. As with the pins 608 and pin holes 504 discussed in step 204, the pins and pin holes 714, 716 discussed herein need not be pins and respectively include screw and screw holes, as appreciated by one of ordinary skill in the art. Additionally, to mount the femoral finishing guide 700 to the first cut surface 420 in the tibial plateau 109, the femoral finishing guide wedge 750 is positioned between the flat base 706 of the femoral finishing guide 700 and the first cut surface 420 in the tibial plateau 109 to prevent the medial compartment 114 of the knee from collapsing. In some embodiments, this step involves selecting the femoral finishing guide wedge 750 among a plurality of femoral finishing guide wedges of different thickness (c.g. about 8 mm, about 9 mm, about 10 mm) along a longitudinal axis 408 of the tibia when positioned on the first cut surface 420 in the tibial plateau 109. In these embodiments, the femoral finishing guide wedge 750 is selected among the plurality of femoral finishing guide wedges based on a dimension of a gap along the longitudinal axis 408 of the tibia between the flat base 706 of the femoral finishing guide and the first cut surface 420.

In an embodiment, after the femoral finishing guide 700 is mounted to the second cut surface 620 and first cut surface 420, the cutting instrument 412 is used to form additional cut surfaces 720, 722 in the medial condyle 108. FIGS. 8A and 8B depict the alignment of the slots 702, 704 through which to pass the cutting instrument 412 and to form the cut surfaces 720 (FIG. 8C) in the medial condyle 108. Another cut surface 722 is not depicted in FIG. 8C because it is posterior to cut surface 720 and out of the field of view of the camera. As shown in FIG. 8B after mounting the femoral finishing guide 700 to the second cut surface 620 and after positioning the femoral finishing guide wedge 750 between the flat base 706 and the cut surface 420 in the tibial plateau 109, the cutting slots 702, 704 are aligned with desired portions of the medial condyle 108 to form the cut surfaces 720, 722.

The cutting instrument 412 is then moved through each of the cutting slots 702, 704 to form the cut surfaces 720 in the medial condyle 108 (FIG. 8C).

### 1.3 Forming Holes in the Cut Surfaces

After steps 202 and 204 forming the cut surfaces in the tibia and femur, one or more holes are formed in the cut surfaces in steps 206 through 212. In one embodiment, the holes formed in the cut surface of the tibia have one or more dimensional ranges, such as a diameter of about 8 mm or in a range from about 6 mm to about 10 mm and/or a depth of about 10 mm (perpendicular to the cut tibial surface) or in a range from about 8 mm to about 12 mm. In an embodiment, these holes are formed in the cut surfaces to accommodate mounting implants to each of the cut surfaces in the tibia and femur in steps 214 and 216. In another embodiment, the holes formed in the cut surface of the femur have a diameter of about 6 mm or in a range from about 4 mm to about 8 mm and a depth of about 11.5 mm or in a range from about 9 mm to about 14 mm. However, the method 200 need not mount implants to the cut surfaces using steps 206 through 212 and instead may use any other method appreciated by one of ordinary skill in the art (e.g. cement to adhere the implants to the cut surfaces) in which case steps 206 through 212 can be omitted.

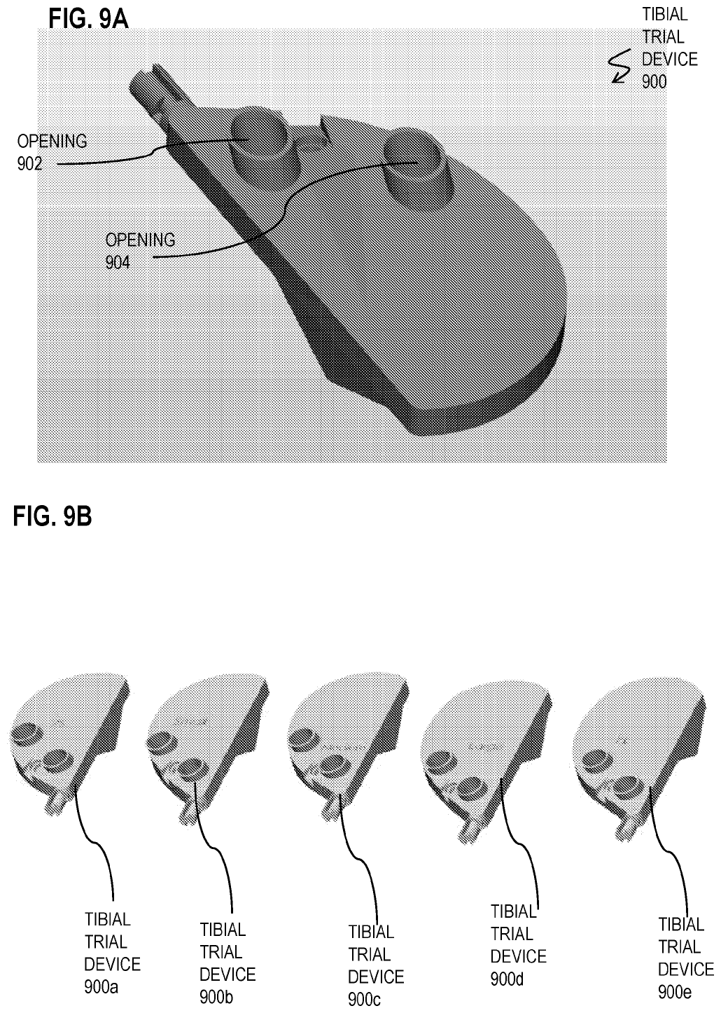

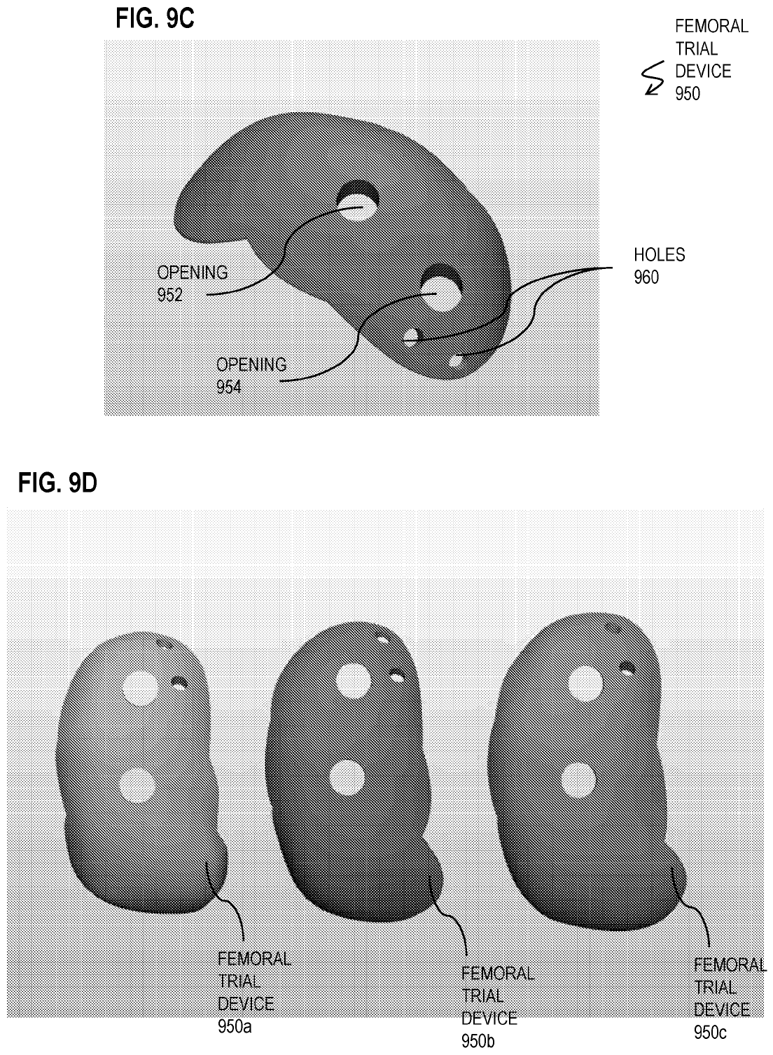

FIGS. 9A through 9D are images that illustrate an example of trial devices 900, 950 of the system 300 that are used to perform steps 206 through 212 of the method 200 of FIG. 2 to form respective holes in the cut surface 420 in the tibial plateau and the cut surfaces 620, 720, 722 in the medial condyle 108, according to one embodiment.

FIGS. 9A and 9B depict a tibial trial device 900 that is used to perform steps 206 and 208. The tibial trial device 900 defines a plurality of openings 902, 904 to form a respective plurality of first holes (not shown) in the first cut surface 420 from step 202. In these embodiments, the tibial trial device 900 has a profile based on a shape of the cut surface 420 of the tibial plateau 109. In some embodiments, the system 300 includes a plurality of tibial trial devices 900a, 900b, 900c, 900d, 900e (FIG. 9B) and in step 206 one of the tibial trial devices is selected to be used in steps 206 and 208. In this example embodiment, the tibial trial device is selected based on which tibial trial device has a profile or surface area which most closely matches the profile of the cut surface 420 and extends on that lateral cut surface 420 out to the medial aspect of the tibia.

In an example embodiments, in step 206 the openings 902, 904 of the tibial trial device 900 are aligned with the cut surface 420 of the tibial plateau 109. In this example embodiment, in step 208 a plurality of first holes (not shown) are then formed in the cut surface 420 by passing a drill through the openings 902, 904 that are aligned with the cut surface 420. In an example embodiment, the openings 902, 904 in the tibial trial device 900 are peg holes that are oriented at a non-zero angle (e.g. about 30 degrees) relative to each other such that the openings 902, 904 are converging relative to each other. The inventors of the present invention recognized that this arrangement enhances the degree that converging pegs of an implant device will secure within the converging holes formed in the cut surface 420.

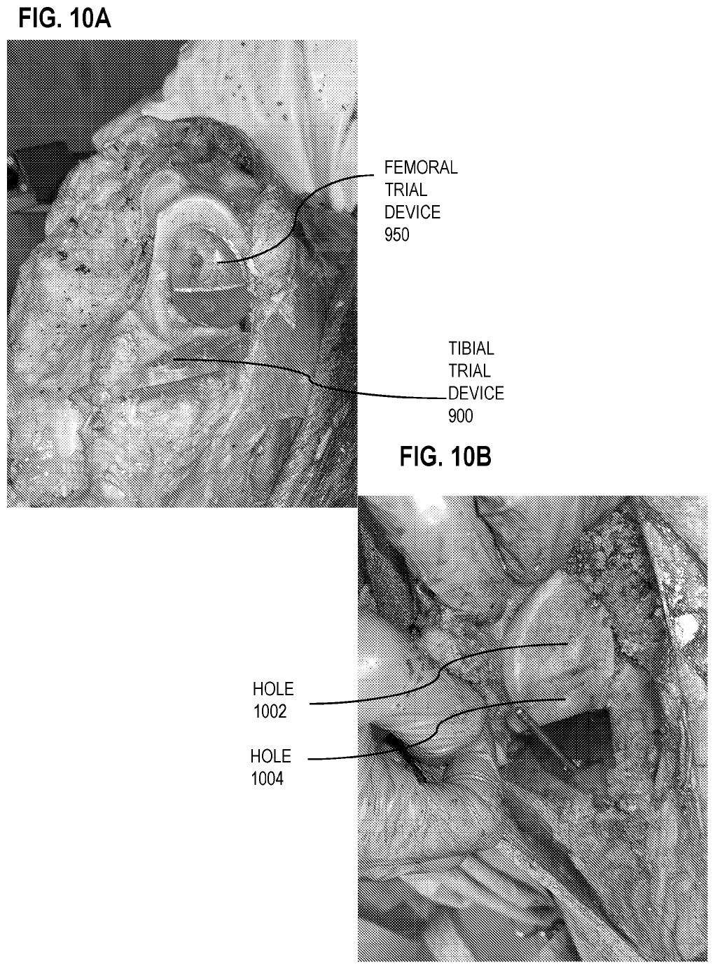

FIGS. 9C and 9D depict a femoral trial device 950 that is used to perform steps 210 and 212. The femoral trial device 950 defines a plurality of openings 952, 954 to form a respective plurality of second holes 1002, 1004 (FIG. 10B) in the cut surfaces 720, 722 from step 204. In these embodiments, the femoral trial device 950 has a profile based on a shape of the cut surfaces 620, 720, 722 of the medial condyle 108. In some embodiments, the system 300 includes a plurality of femoral trial devices 950a, 950b, 950c (FIG. 9D) and in step 208 one of the femoral trial devices is selected to be used in steps 210 and 212. In this example embodiment, the femoral trial device is selected based on which femoral trial device has a profile or surface area which most closely matches the profile of the cut surfaces 620, 720, 722. A pair of converging pin holes 960 are also provided for fixation of the femoral trial device 950 to the cut surfaces 720, 722.

In an example embodiments, in step 210 the openings 952, 954 of the femoral trial device 950 are aligned with the cut surfaces 720, 722 of the medial condyle 108. In this example embodiment, in step 212 a plurality of second holes 1002, 1004 are then formed in the cut surfaces 620, 720 by passing a drill through the openings 952, 954 that are aligned with the cut surfaces 620, 720. In an example embodiment, the openings 952, 954 in the femoral trial device 950 are peg holes that are oriented at a non-zero angle relative to each other such that the openings 952, 954 are converging relative to each other. The inventors of the present invention recognized that this arrangement enhances the degree that converging pegs of an implant device will secure within the converging holes formed in the cut surfaces 620, 720.

In an embodiment, the second cut surfaces 620, 720, 722 in the medial condyle based on step 204 are three second cut surfaces 620, 720, 722 that are angled relative to each other. In an example embodiment, in steps 210 and 212 the openings 952, 954 in the femoral trial device 950 are aligned with and holes are formed in two of the three second cut surfaces 620, 720. However, in other embodiments, in steps 210 and 212 the openings 952, 954 are aligned with two other second cut surfaces.

### 1.4 Mounting Implants to the Cut Surfaces in Tibia and Femur

In an embodiment, after forming the holes in the cut surfaces of the tibia and femur, implants arc mounted to these cut surfaces. For purposes of this description, the implants are “mounted” to the cut surfaces using one of a multiple of techniques, including but not limited to cement (polymethylmethacrylate or PMMA), creating so-called cemented fixation; cementless fixation (e.g., uses special bone ingrowth surfaces for the bone to attach); or having a root to the implant (especially the tibia) so it forms a T-shape and then placing a screw from an exterior bone surface that would thread into the bottom of the T-shaped implant and thus lock the implant into place. Although the embodiments herein discuss mounting the implants to the cut surfaces of the tibia and femur using peg holes in which pegs of the implants are secured, any method appreciated by one of ordinary skill in the art can be used to mount the implants to the cut surfaces of the tibia and femur. In an example embodiment, these alternate embodiments could involve the use of cement to mount the implants to the cut surfaces in the tibia and femur, in which case steps 206 through 212 could be omitted and/or replaced with steps of applying cement to the cut surfaces and/or inner surfaces of the implants that are mounted to the cut surfaces.

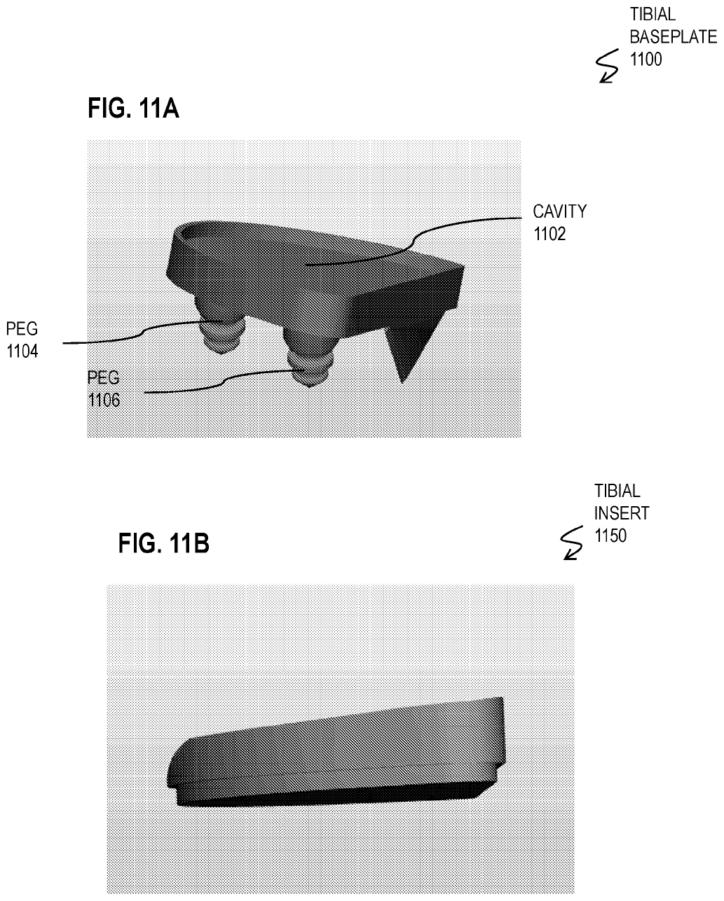

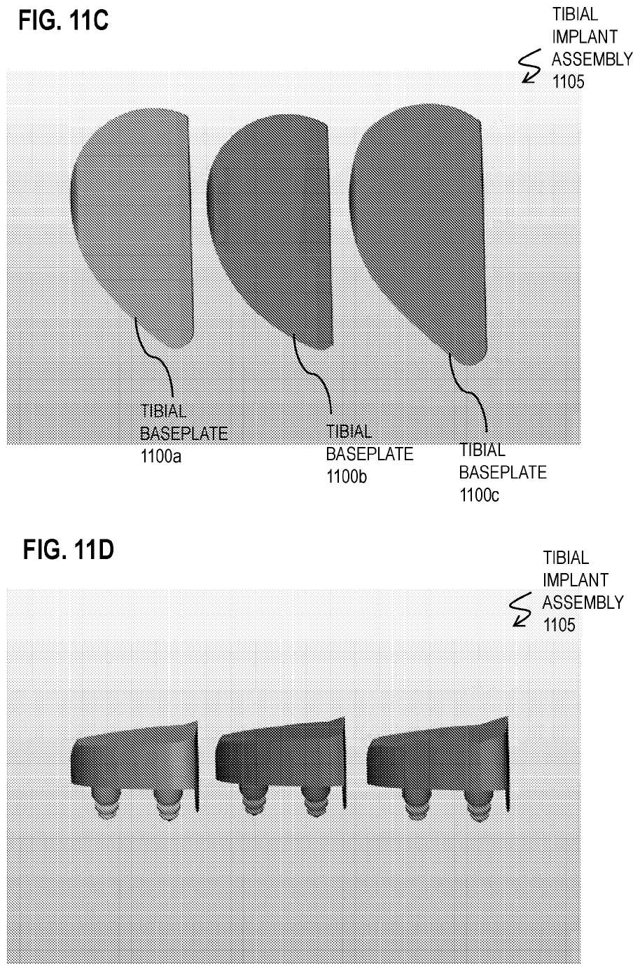

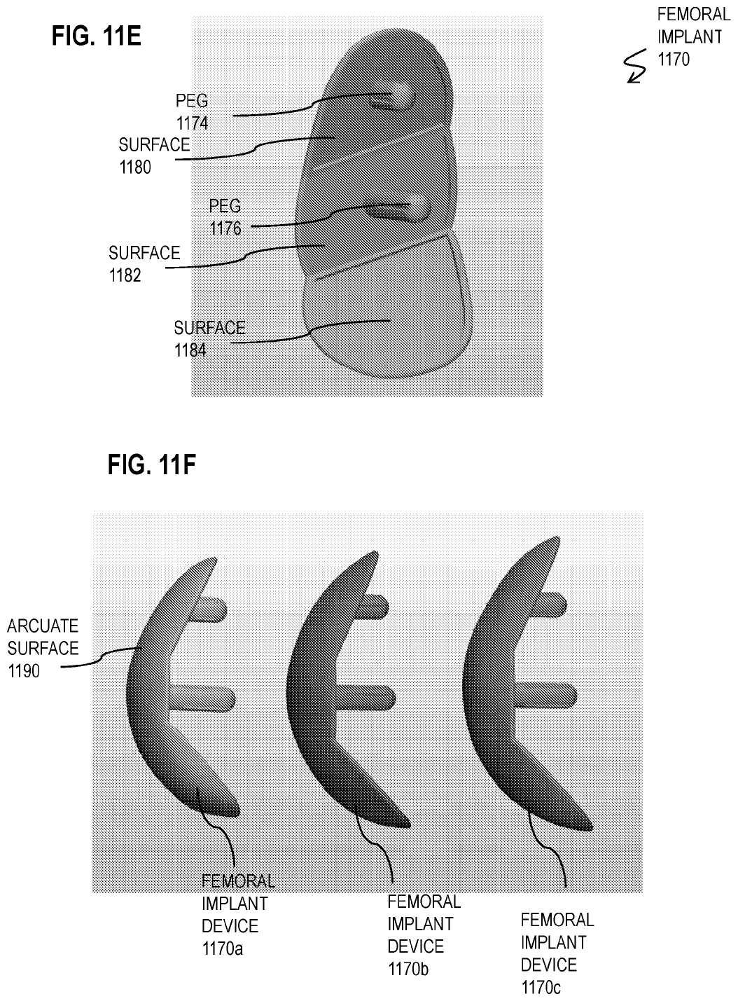

FIGS. 11A through 11F are images that illustrate an example of implant devices 1100, 1150, 1170 of the system 300 that are used to perform steps 214 and 216 of the method 200 of FIG. 2. In an embodiment, steps 214 and 216 involving mounting implant devices 1100, 1150, 1170 to the respective holes formed in the tibial plateau 109 and medial condyle 108 in steps 206 through 212.

The first implant device that is implanted in step 214 to the cut surface 420 of the tibial plateau 109 will now be discussed. In an embodiment, the tibial baseplate 1100 is an implant device for the cut surface 420 of the tibial plateau 109. The tibial baseplate 1100 includes a plurality of pegs 1104, 1106 configured to be inserted in the plurality of first holes formed in step 208 to securely mount the tibial baseplate 1100 to the first cut surface 420 of the tibial plateau 109. As further shown in FIGS. 11A and 11B, the tibial baseplate 1100 defines a cavity 1102 that is sized such that a tibial insert 1150 can be placed and secured within the cavity 1102. In an example embodiment, for cemented applications the tibial baseplate 1100 is made of a Co/Cr/Mo alloy that is standard in the orthopedic industry and for uncemented applications the tibial baseplate 1100 is made from Ti6A14V material. In another example embodiment, the tibial insert 1150 is made of UHMWPc material with moderate cross-linking and some oxidation stabilizing agent like tocopherol (Vitamin E). In yet another embodiment, one or both of the femur and tibial inserts from engineering plastics (e.g. in the PEEK family of polymers). In another embodiment, the plurality of pegs 1104, 1106 are oriented at a non-orthogonal angle (e.g. about 45 degrees) relative to the tibial baseplate 1100. In an example embodiment, the tibial baseplate pegs 1104, 1106 are about 11.6 mm in length or in a range from about 9 mm to about 14 mm. In an example embodiment, both pegs 1104, 1106 have the same orientation and length.

A tibial implant assembly 1105 includes the combination of the tibial baseplate 1100 and tibial insert 1150 placed and secured within the cavity 1102 of the tibial baseplate 1100. In one embodiment, FIGS. 11C and 11D depict different tibial implant assemblies 1105 where each tibial baseplate 1100a, 1100b, 1100c has a respective tibial insert 1150 secured within the cavity 1102 of each tibial baseplate 1100a, 1100b, 1100c. In an embodiment, in step 214 a tibial baseplate 1100 is selected among a plurality of tibial baseplates 1100a, 1100b, 1100c (FIGS. 11C and 11D) with different dimensions. In one embodiment, the tibial baseplate 1100 is selected based on which tibial baseplate 1100 has the profile or dimensions that most closely fits the cut surface 420 in the tibial plateau 109. In an example embodiments, the dimensions of the cut surface 420 can vary based on an age of development of the mammal (e.g. horse). In this embodiment, a respective tibial insert 1150 is then secured within the cavity 1102 of the selected baseplate 1100 to form the tibial implant assembly 1105.

The second implant device that is implanted in step 216 to the cut surfaces of the medial condyle 108 will now be discussed. In an embodiment, the second implant device is a femoral implant device 1170 depicted in FIG. 11E. In one embodiment, the femoral implant device 1170 includes a plurality of pegs 1174, 1176 configured to be inserted in the plurality of second holes 1002, 1004 (FIG. 10B) to securely mount the femoral implant device 1170 to the second cut surfaces 720, 722 of the medial condyle 108. In an example embodiment, the middle cut femoral surface 720 is perpendicular to the long peg 1176 on the femoral implant 1170 and the shorter peg 1174 is parallel to the long peg 1176. As further depicted in FIG. 11E, in one embodiment the femoral implant device 1170 has an inner surface that defines a plurality of surfaces 1180, 1182, 1184 that are angled relative to each other. In one example embodiment, the plurality of pegs 1174, 1176 extend from two of these surfaces 1180, 1182. However, in other embodiments the pegs can extend from all of these surfaces 1180, 1182, 1184 or two different surfaces than depicted in FIG. 11E. In an example embodiment, the angles between the surfaces 1180, 1182, 1184 are about equal to an angle between the cut surfaces 620, 720, 722 of the medial condyle. The inventors of the present invention recognized that this facilitates step 216 in securing the femoral implant device 1170 to the medial condyle 108. As further shown in FIG. 11F, the femoral implant device 1170 has an outer surface 1190 that has an arcuate shape based on an arcuate shape of the medial condyle 108 that was removed in step 204. In an example embodiment, the femoral implant device 1170 is made of similar material as the tibial insert 1150 that was previously discussed.

In an embodiment, in step 216 a femoral implant device 1170 is selected among a plurality of femoral implant devices 1170a, 1170b, 1170c (FIG. 11F) with different dimensions. In one embodiment, the femoral implant device 1170 is selected based on which femoral implant device 1170a, 1170b, 1170c has the profile or dimensions that most closely fits the cut surfaces 620, 720, 722 in the medial condyle 108. In an example embodiments, the dimensions and/or angles between the cut surfaces 620, 720, 722 can vary based on an age of development of the mammal (e.g. horse).

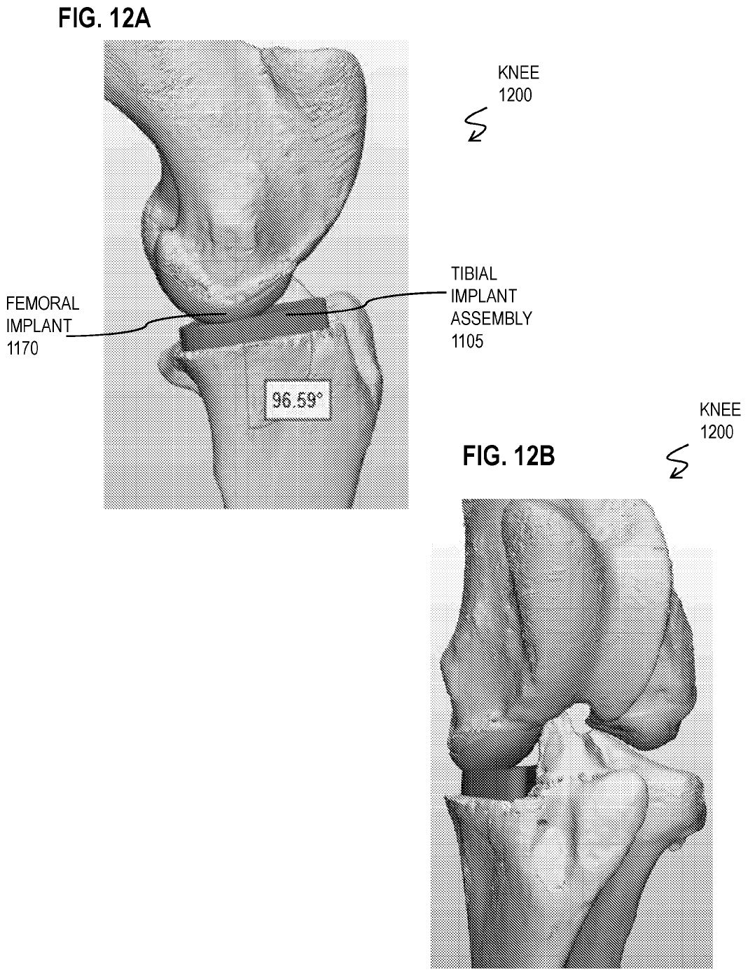

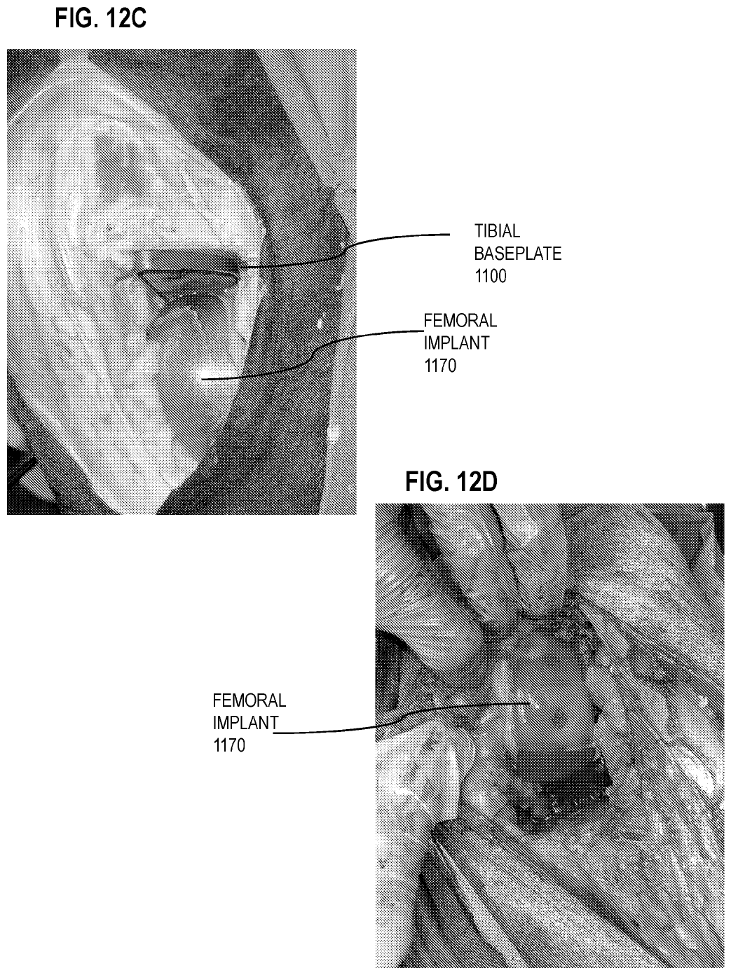

FIGS. 12A through 12D are images that illustrate an example of the implant devices 1100, 1150, 1170 of FIGS. 11A through 11F being used in steps 214 and 216 to mount the implant devices 1100, 1150, 1170 to the tibial plateau 109 and medial condyle 108, according to one embodiment. FIGS. 12A and 12B show the knee joint after performing steps 214 and 216, namely where the tibial baseplate 1100 (and tibial insert 1150) is mounted to the cut surface 420 in the tibial plateau 109 and the femoral implant 1170 is mounted to the cut surfaces 620, 720, 722 in the medial condyle 108. In an example embodiments, FIGS. 12C and 12D show images that also illustrate the femoral implant 1170 and tibial baseplate 1100 mounted to the respective cut surfaces in the medial condyle 108 and tibial plateau 109. This advantageously results in a partial knee replacement of the medial compartment 114.

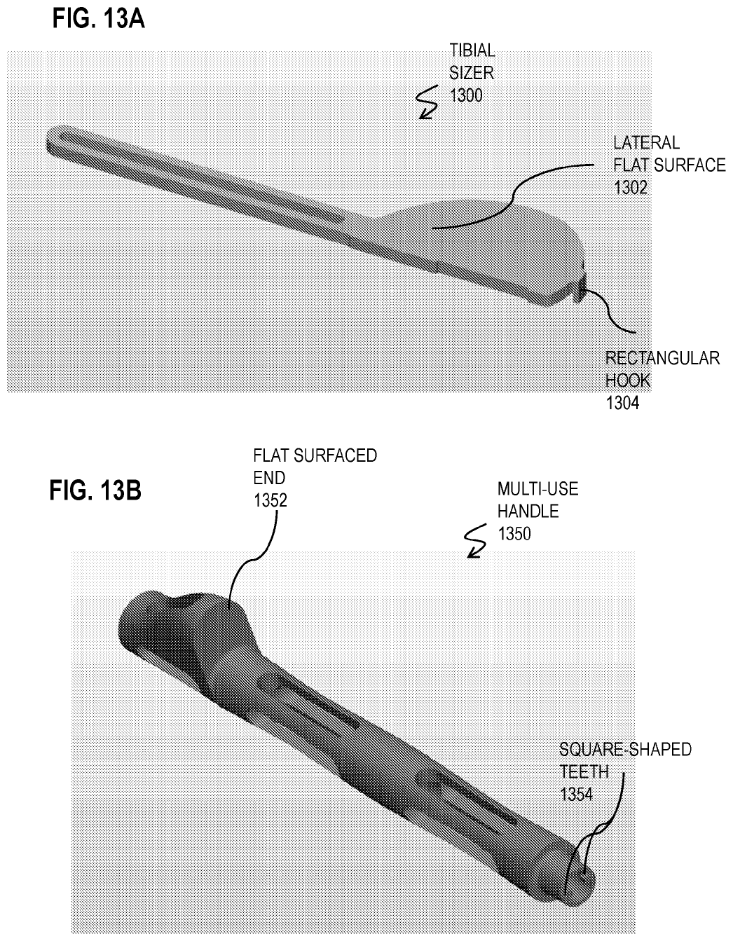

Another component of the system 300 will now be discussed that can be used in mounting the tibial insert to the cut surface in the tibial plateau (step 214). FIG. 13A is an image that illustrates an example of a tibial sizer 1300 used to determine an optimal size of a tibial insert 1150 to be used in performing steps of the method of FIG. 2, according to one embodiment. In an embodiment, multiple tibial sizers 1300 (e.g. five) with different sizes are provided that share the same contour/shape with tibial implants 1150 that are available in the current kit. In one embodiment, the tibial sizer 1300 was designed to help surgeons determine the optimal size of the tibial implant 1150 for the patient in performing step 214. Alignment in the medial-lateral and cranial-caudal directions were guided by the lateral flat surface 1302 and the rectangular hook 1304 at the most caudal margin of the sizer 1300, respectively. After two femoral cuts using the femoral finishing guide 700, the tibial sizer 1300 was positioned on the cut surface sitting flush in the tibial plateau 109. The featured hook 1304 was positioned at the caudal edge of the tibial plateau 109 (back of the tibia), meanwhile, the lateral surface 1302 of the sizer 1300 should also be flush with the medial surface of the tibial plateau 109 created by the tibial vertical cut. When the cranial edge of the sizer 1300 (front side that is closer to the handle) aligns with the cranial edge of the tibial plateau 109 (a slight under-hang is desired), the size of the tibial implant 1150 is determined as the same size of the tibial sizer 1300.

FIG. 13B is an image that illustrates an example of a multi-use handle 1350 used to facilitate insertion and extraction of wedges in performing steps of the method of FIG. 2, according to one embodiment. In one embodiment, the multi-use handle 1350 was used during one or more steps of the method 200. The tool 1350 features a simple twist lock design that is compatible with all the wedges (femoral resection guide wedges 550, femoral finishing guide wedges 750) and the tibial trial devices 900. The inventors of the present invention designed the multi-use handle 1350 to facilitate the insertion and the extraction of the wedges and tibial trials. The smaller end with two square-shaped teeth 1354 can be assembled with instruments (the wedges and tibial trials) that are featured with a cylindrical knob. Once the multi-use handle 1350 was inserted and linked with aforementioned instruments, a 90° counterclockwise twist should lock the tool and the instrument in place. By hammering the flat-surfaced end 1352, the tool facilitates the insertion of the instruments. By hammering the flat surfaced end 1352 on the slope end, the tool facilitates the extraction of the instruments.

### 2. Example Embodiments

Some example embodiments will now be discussed which relate to the design and modelling of the implants used in steps 214 and 216. Aseptic loosening and tibial implant wear are the most common modes of failure in fixed bearing UKA, which could result from poor polyethylene formulation, tibial implant conformity, and implant malalignment. As advances have been made in tibial implant material, implant wear issues have been significantly improved. However, aseptic loosening and tibial implant wear remain common issues in UKA with fixed bearing, which suggests underlying mechanical issues in the implant design.

Conformity, defined as the femoral radius divided by the tibial radius in each plane, is one of the most important factors in UKA design as more conforming designs can reduce contact stress and thus less tibial implant wear. However, increased conformity could result in constraints on joint movement, increased contact stress if implant malalignment occurs, increased wear due to easier wear particle entrapment between the articular surfaces, increased component interface stresses, and micro-motion. To date, no literature on UKA design for large animal stifle joints has been reported. The current femoral implant articular surface (e.g. arcuate surface 1190 in FIG. 11F) follows the mean shape of the medial femoral condyle generated by statistical shape modeling (SSM), while the geometry of the tibial implant (e.g. tibial implant assembly 1105) has yet to be optimized. Therefore, one of the challenges of equine UKA design is to determine the optimal tibial implant conformity to balance the advantage of reduced stresses and the disadvantage of constraints on joint kinematics. Intuitively, concerns were raised regarding if the conventional ultra-high molecular weight polyethylene (UHMWPE) commonly used in human tibial implants would be able to withstand the force in the equine stifle joint as the weight of a horse is much greater, and the force of muscle contractions associated with the stifle joint during gait is much higher than that of in the human knees. Knee replacement designs with different conformities are often tested using knee simulator machines. However, testing a single design can be costly and time-consuming. Finite element analysis (FEA) is one method to evaluate the stress distribution on the tibial implant by simulating how the finite element (FE) models of the implants would behave mechanically under various loading conditions. Such a method is able to produce contact stress predictions for various designs with the same motion and load inputs in a relatively short period of time.

In a present study, FEA was utilized to assess the influence of different conformities under cyclic normal loading at approximate 150° extension of the equine femorotibial joint. Testing of the FE models was divided into three groups. The first group blanketed a wide range of conformities with constant coronal and sagittal radii tibial implants in combination with a constant coronal radius femoral implant. The second group explored various permutations of varying conformities in the coronal plane, which consisted of two different radii alongside the anatomy-based femoral implant. The third group consisted of tibial implants with 3 different sagittal radii paired with the anatomy-based femoral implant. The first group aimed to assess the performance of designs with symmetric profiles. The second group investigated the effect of different combinations of asymmetric coronal conformities. The third group evaluated the influence of sagittal conformity. Contact pressure, contact area, and plastic deformation depth in the tibial bearing were evaluated in all three groups.

3D models of the femoral implant (e.g. femoral implant 1170) and tibial baseplate (e.g. tibial baseplate 1100) were generated by previously developed statistical shape models in 3-matic®. All computer-aided design (CAD) models were designed in Solidworks® (Dassault Systèmes SOLIDWORKS® Corp., Massachusetts).

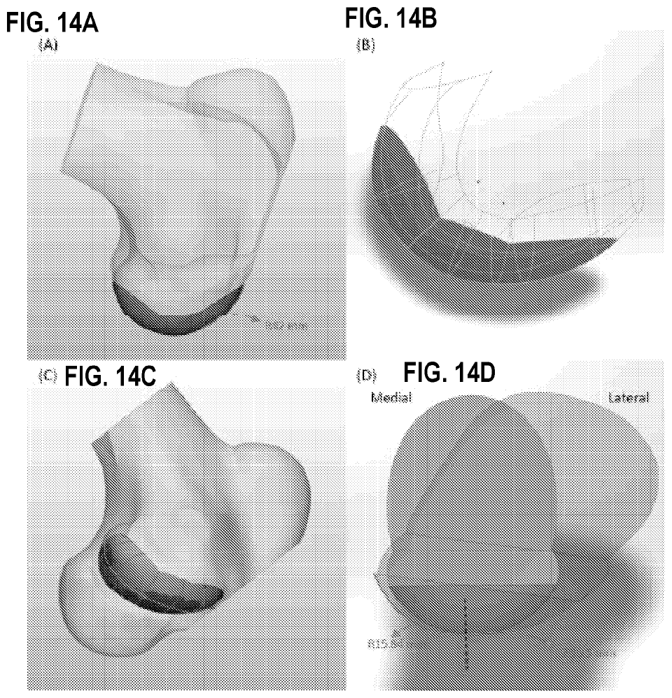

The equine UKA femoral implant was created based on the 3D model of the mean femur shape generated by statistical shape modeling in 3-matic, and the ideal resurfaced distal femoral condyle was acquired as shown in FIG. 14A. The cut-off from the medial femoral condyle 114 was exported to Solidworks® and used to serve as a guide to constructing the femoral implant (e.g. femoral implant 1170) geometry with a series of B-splines in the coronal planc 406 and sagittal planc 404. This was designed to maximize the fitting of the femoral implant (e.g. femoral implant 1170) to the native medial femoral condyle 114, as shown in FIG. 14B. As the UKA aims to restore normal knee kinematics by mimicking the native knee anatomy, the sagittal geometry of the femoral implant (e.g. femoral implant 1170) often follows the patient's femoral condylar geometry, and the coronal geometry is modified to optimize the mechanical behaviors and functions. The femoral implant (e.g. femoral implant 1170) was approximated using 3 varying radii in the sagittal profile and various radii in the coronal profile at multiple locations along the sagittal profile, as shown in FIG. 14C.

For the first group, a simplified version of the anatomy-based femoral implant (e.g. femoral implant 1170) was constructed using a constant coronal radius and a constant sagittal radius to investigate the contact pressure at the maximum femorotibial extension. The 42 mm sagittal radius, which corresponds to the craniodistal segment of the femoral implant, was utilized. The constant coronal radius of the simplified femoral implant was established based on the average curvature of 17.4 mm radius, measured at multiple locations on the femoral condyle along the craniocaudal direction. For groups two and three, the anatomy-based femoral implant was utilized. From medial to lateral, the 2 coronal radii were 15.84 mm and 33 mm (FIG. 14D).

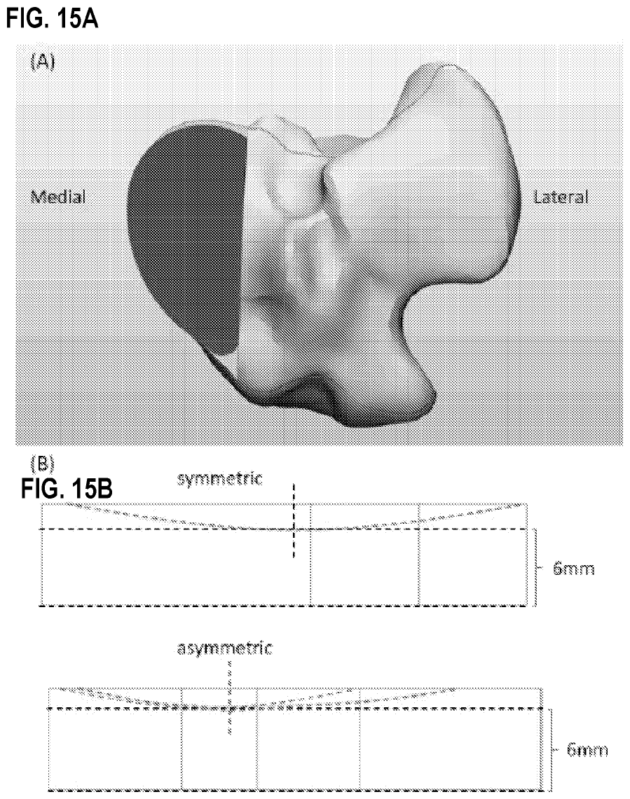

Similarly, the equine UKA tibial implant (e.g. implant assembly 1105, tibial baseplate 1100, tibial insert 1150) was created based on the 3D model of the mean tibial shape generated by statistical shape modeling. The transverse profile of the tibial implant was guided by the shape of the tibial osteotomy, maximizing the coverage of the cortical bone (FIG. 15A). The minimum thickness of all tibial implants tested was 6 mm, measured from the lowest articular point to the bottom surface of the bearing.

For group one, a matrix of 16 permutations of constant radius tibial implants with 4 levels of conformities in either the coronal plane 406, sagittal plane 404 or both was created. All conformities were calculated based on the dimensions of the simplified femoral implant used in group one. The second group evaluates bearings with asymmetric coronal profiles consisting of 2 conformities: the medial and the lateral, where the separation was determined by the lowest contact point when the stifle joint is at 150° in extension. As shown in a previous report that moderate sagittal conformity is desirable for reducing wear, the matrix for group two in the current study evaluated 9 combinations of conformities on the medial and lateral portions of the coronal profile in combination with the anatomy-based femoral implant. The sagittal conformity was fixed at 0.5 (e.g., a sagittal radius of 84 mm) (FIG. 15B). Group three tested three designs consisting of 0.25 medial and 0.75 lateral coronal conformity with various sagittal conformities: 0.25, 0.5, and 0.75. Conformity of 0 represents a flat profile in the corresponding plane. The maximum conformity used in the current study was 0.75, as very high conformity affects the proper function of the cruciate ligaments and offers diminishing returns for reducing wear.

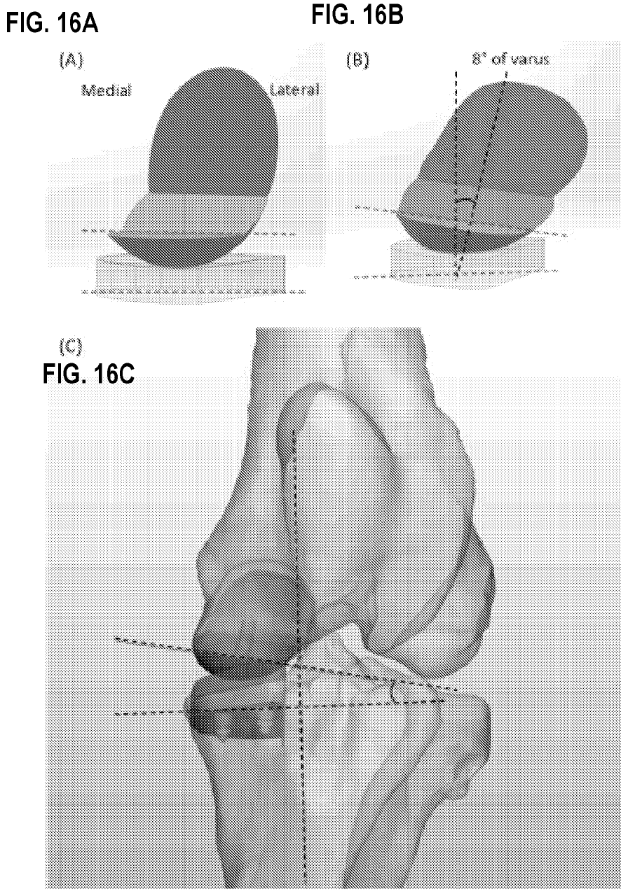

Matching femoral implants and tibial implants were assembled and aligned in Solidworks®. It was estimated that at the maximum extension of the stifle joint, the craniodistal bone-implant interface of the femoral implant was paralleled to the bottom surface of the tibial bearing. As the simplified femoral implant used in group one was symmetric in the coronal plane 406 and the contact was only tested at the maximum extension, the femoral implant was positioned without varus-valgus angulation as shown in FIG. 16A. In group two, the femoral implant was positioned at 8° of varus sitting at the lowest point of the tibial bearing as shown in FIGS. 16B and 16C. The assemblies were then exported as Step files and analyzed in Abaqus® 6.24 (Simulia, Providence, RI).

The cobalt-chromium femoral implants were modeled as linear elastic isotropic bodies using Young's modulus of 195,000 MPa and a Poisson's ratio of 0.3. The tibial bearing was modeled as an elastic-plastic material GUR 1020 with a modulus of elasticity of 900 MPa and a Poisson's ratio of 0.46. A penalty-based contact condition was employed between the implant-bearing interface with a friction coefficient of 0.04. The femoral implant was meshed by using 10-node tetrahedral elements with a mean edge length of 1.5 mm. The tibial bearing was meshed by using linear hexahedral elements with a mean edge length of 1.2 mm.

Joint loading in the equine stifle has yet to be fully explored. Two equine medial femoral condyle models were mechanically loaded with 6000N and 8000N, which were estimated based on the assumption that the joint loads in the cquinc forclimbs have approximately the same magnitude as that of the hindlimbs during galloping. Due to the lack of knowledge of the joint load distribution in the medial and lateral equine stifles, a 7000 N axial load was used as the maximum value in the medial compartment in the current study.

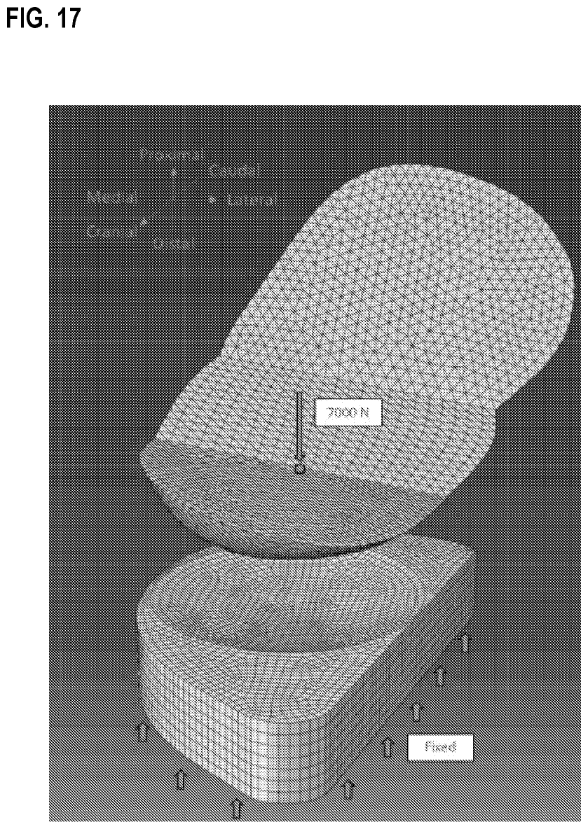

The axial load was modeled as a triangular waveform cyclic loading with 4 cycles fluctuating between 0 and 7000 N, which was applied perpendicular to the craniodistal surface at the mid-point of the intersection line of the distal and the chamfer surface. The femoral component was controlled to only translate in the proximal-distal direction, and the bottom of the tibial bearing was fixed (FIG. 17). The contact area, contact pressure, and plastic deformation for each FE model was evaluated and compared to FE models with different conformities within the same testing group.

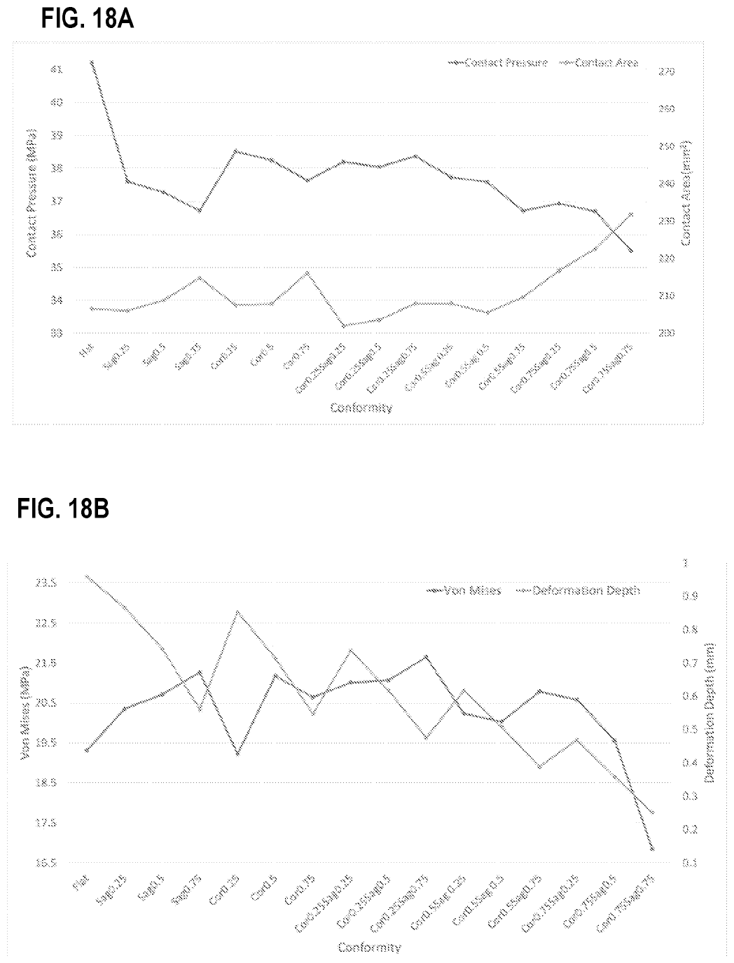

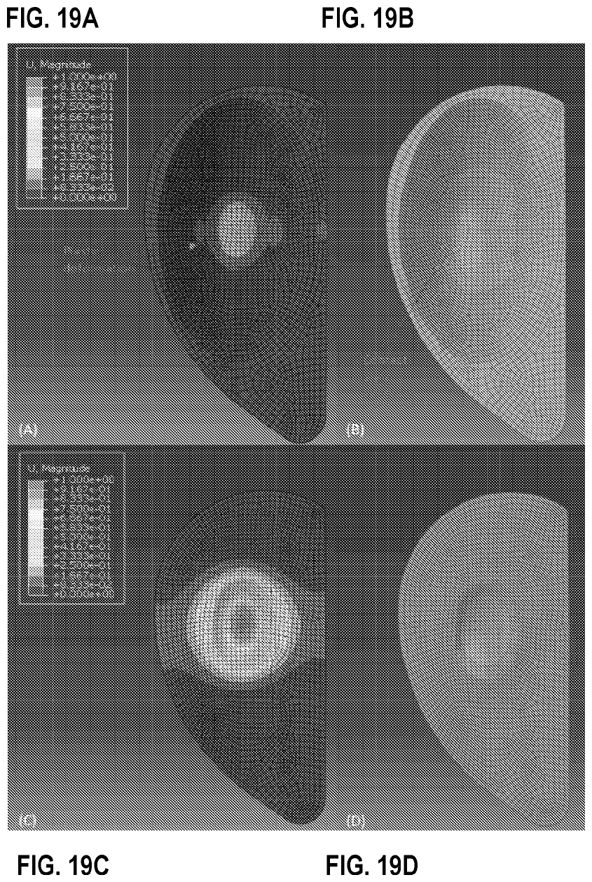

In group one, the flat bearing showed the smallest contact area, highest contact pressure, and the largest plastic deformation depth. The bearing with the highest conformities in both coronal 406 and sagittal planes 404 showed the largest contact area, lowest contact pressure and Von Mises stress, and the least deformation (FIGS. 18A and 18B). The overall trend of higher contact area with lower contact pressure was observed across the group. Interestingly, when the coronal conformity is 0.25, only a slight increase in the contact area and a small change in contact pressure were observed for sagittal conformities from 0.25 to 0.75. With conformities in both planes being moderate (0.5) or high (0.75), the trend became more apparent. When comparing deformation depth with conformity in either plane, the deformation decreased as the conformity increased, suggesting deformation sensitivity to conformity in both planes. Moreover, the only design without high conformity in either plane that showed deformation equal to or less than 0.5 mm is the coronal 0.5 and sagittal 0.5. A visual comparison of the bearing deformation and contact area at the maximum contact pressure is shown in FIGS. 19A through 19D.

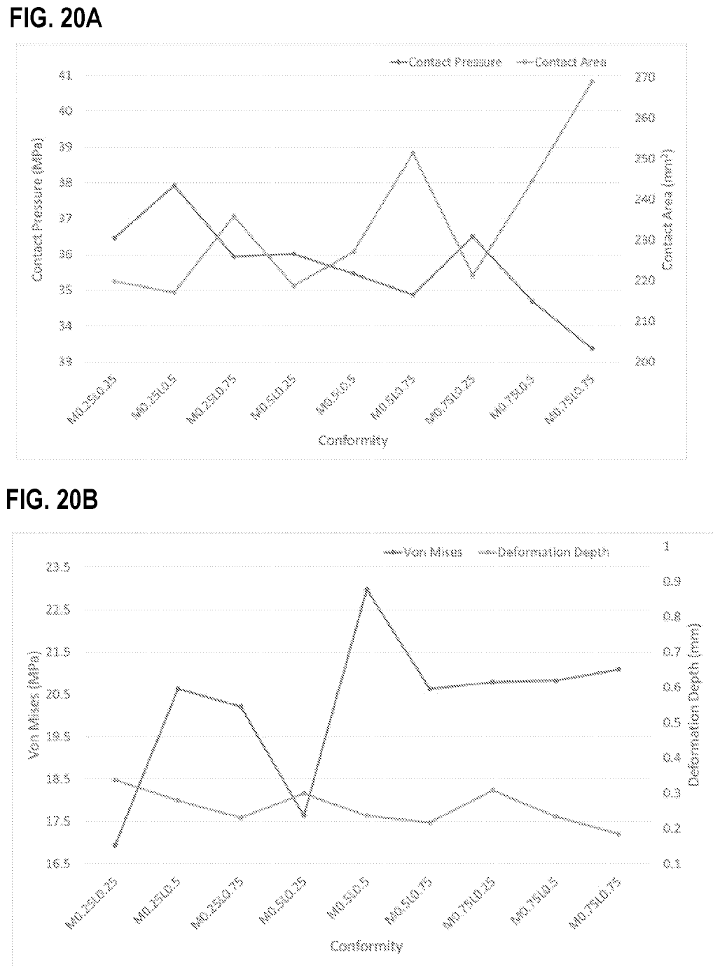

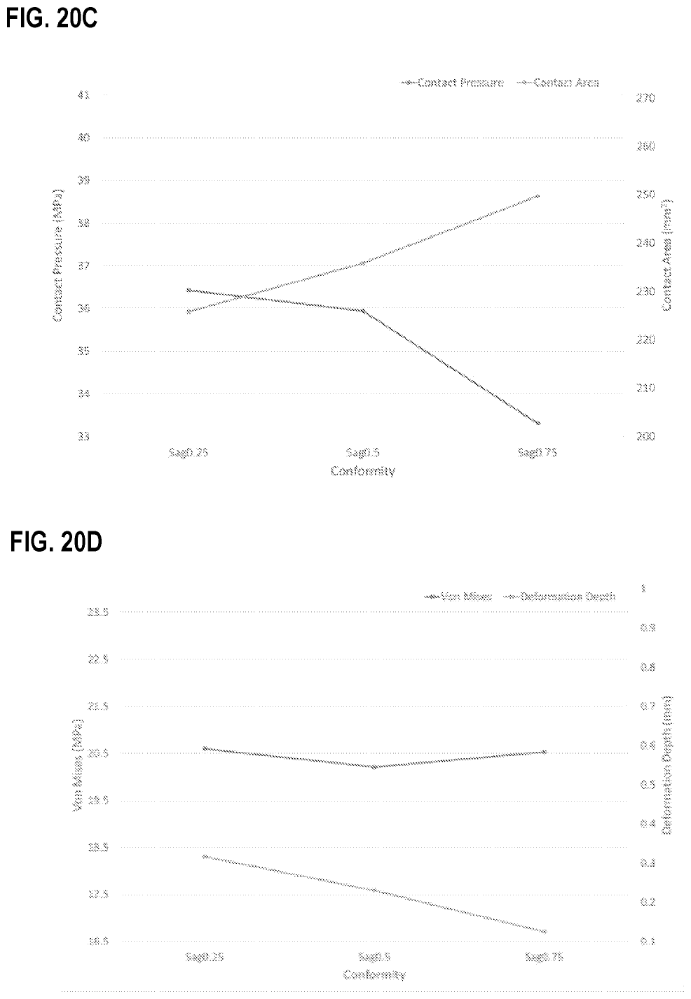

Group two demonstrated nine different combinations of coronal conformity in the medial and lateral portions (FIGS. 20A and 20B). For instance, “M0.25L0.5” denotes a combination of 0.25 coronal conformity in the medial portion and 0.5 in the lateral portion. All designs showed similar peak contact pressure. There was a slow increase in the maximum contact area. Conformity M0.75L0.75 produced the largest contact area and the lowest contact pressure in group two. Similarly, plastic deformation depth ranged from 0.185 mm to 0.338 mm. All the designs with moderate or high conformity in either direction experienced less than 0.3 mm deformation depth.

Group three evaluated three designs with different sagittal conformities when coronal conformity was fixed at M0.25L0.75 (FIGS. 20C and 20D). Similarly, conformity of 0.25 in the sagittal plane was abbreviated to Sag0.25. The maximum contact area increased by approximately 14 mm², while the peak contact pressure decreased by approximately 3.1 MPa from Sag0.25 to Sag0.75. Approximately 0.4 MPa 0.2 mm differences were shown in peak Von Mises stress and plastic deformation depth within the group.

The current study used FEA to investigate the influence of tibial sagittal and coronal conformity on contact pressure, contact area, and tibial implant deformation in equine UKA. The designs and the FE models were representative of idealized joint loadings and alignment in the equine stifle. The FE models were divided into three groups, where group one consisted of a simplified femoral implant with a constant coronal radius along with tibial implants with various constant conformities in coronal and sagittal planes, while group two explored tibial implant designs with varying conformities in the coronal plane paired with the femoral implant that mimics the mean geometry of the native femoral condyle produced by SSM. Group three investigated the influence of sagittal conformity with anatomy-based femoral implants and varying coronal radius tibial bearings.

The FEA results showed general agreement with previous computational studies that increased conformity results in a larger contact area, lower contact pressure, and less bearing deformation. In group one, the flat bearing showed a slightly higher maximum contact area than Cor0.25Sag0.25 and Cor0.25Sag0.5. This could possibly be explained by the large deformation depth. As the femoral implant experiences “bedding in” or creep deformation, the contact area increases. In addition, 4 designs (Cor0.25Sag0.5, Cor0.25Sag0.75, Cor0.5Sag0.25, and Cor0.5Sag0.5) all showed similar maximum contact arca and deformation depth, which could suggest that these 4 designs did not reach maximum plastic deformation after 4 cycles of 7000 N loading; the maximum contact area was the exact shape of the femoral implant imprinted in the bearing due to creep. In designs with higher conformities, such as Cor0.75Sag0.25, Cor0.75Sag0.5, and Cor0.75Sag0.75, the maximum contact area increased at a much higher rate with sagittal conformity, while the plastic deformation depth and the contact pressure decreased with sagittal conformity. This resulted from the increased conformity and contact area with the design.

Within group two, no significant change in the contact pressure and deformation depth occurred, as the highest and the lowest contact pressure was 37.92 MPa and 33.37 MPa observed in M0.25L0.5 and M0.75L0.75, respectively. The largest and the smallest deformation depth were found to be 0.338 mm and 0.185 mm in M0.25L0.25 and M0.75L0.75, respectively. This could be explained by “diminishing return” as a result of moderate sagittal conformity of 0.5. Though group two did not show much variation in the results within the group, group two performed much better than group one. The relatively weakest performance in the second group was produced by the lowest level of conformity M0.25L0.25, and it outperformed every design except Cor0.75Sar0.75 in group one in peak contact pressure and maximum deformation depth. In addition, on paper, even with two low coronal conformity levels, it demonstrated the third largest contact area just behind the two highest conformity combinations, Cor0.75Sar0.5 and Cor0.75Sar0.75. However, one major contributor to the superior performance in group two was the paired anatomy-based femoral implant. With the same thickness in the craniodistal portion of the femoral implant, the anatomy-based geometry offered a larger implant interface, which intuitively increased the contact area and reduced contact pressure. Therefore, though not completely unexpected, the findings indicated that anatomy-based femoral implant paired with asymmetric coronal conformity provides a larger contact area, lower contact pressure, and less early deformation than the single radius conformity designs.

Group three evaluated the influence of sagittal conformity. Compared to changes seen in group two, group three showed similar ranges of contact pressure, contact area, and deformation depth. The reason why coronal conformity M0.25L0.75 was chosen in group three was that it offered similar performance compared to other designs within group two. Moreover, the tibia abducts approximately 6° during flexion, which increases the contact area on the lateral portion of the medial compartment in the equine stifle. Thus, a more laterally conforming coronal profile design was chosen. The findings in group three indicated that the designs might have reached “diminishing returns” with the coronal conformity as the bearing performance did not show much improvement with higher sagittal conformity.

One of the main limitations of the current study was the lack of joint kinematics as inputs. The current study explored the influence of tibial bearing conformity at a static position. In human knees, the impact of implant design on kinematics has been well documented through experimental and computational studies. As was discussed in numerous studies, striking a balance between high conformity and restoring normal joint kinematics remains a major challenge.

One limitation of the present study was joint load input. If a force distribution in the stifle is proportional to that of the forelimb with a ratio of 60% in the forelimb during walking, the joint load in the medial femorotibial compartment in a 500 kg horse would be approximately 3300 N to 4500 N, which is much lower than what was used in the current study. Furthermore, there was no AP control force or IE control torque, or any muscle forces in the present study, which are important for producing accurate kinematics and implant interactions. Therefore, further investigations into the joint loads in the medial femorotibial joint in horses are needed.

Though idealized geometries were used in the present study, they still provide valuable insight into the effect of conformity on implant performance. The findings from the current study demonstrated anatomy-based femoral implant coupled with a tibial implant consisting of asymmetric coronal conformity in the medial-lateral direction and moderate sagittal conformity could provide competitive results in comparison to higher conformity designs. Higher conformity in either direction offered little benefits. Future studies need to assess the impact of higher conformity on joint kinematics compared to the design with moderate conformities. Furthermore, a better understanding of the creep and wear rate would be beneficial for future implant design.

### 3. Alternatives, Deviations and Modifications

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

Notwithstanding that the numerical ranges and parameters setting forth the broad scope are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Thus, a value 1.1 implies a value from 1.05 to 1.15. The term “about” is used to indicate a broader range centered on the given value, and unless otherwise clear from the context implies a broader range around the least significant digit, such as “about 1.1” implies a range from 1.0 to 1.2. If the least significant digit is unclear, then the term “about” implies a factor of two, e.g., “about ×” implies a value in the range from 0.5× to 2×, for example, about 100 implies a value in a range from 50 to 200. Moreover, all ranges disclosed herein are to be understood to encompass any and all sub-ranges subsumed therein. For example, a range of “less than 10” for a positive only parameter can include any and all sub-ranges between (and including) the minimum value of zero and the maximum value of 10, that is, any and all sub-ranges having a minimum value of cqual to or greater than zero and a maximum value of equal to or less than 10, e.g., 1 to 4.

What is claimed is:

1. A system for performing a unicompartmental knee arthroplasty in a knee of a mammal, comprising:
at least one first cutting device configured to cut a portion of a plateau of a tibia to form a plurality of first cut surfaces in the tibial plateau;
at least one second cutting device configured to cut a portion of a medial condyle of a femur to form a plurality of second cut surfaces of the medial condyle;
at least one first trial device defining a plurality of openings to form a respective plurality of first holes in at least one of the first cut surfaces that are aligned with the plurality of openings;
at least one second trial device defining a plurality of openings to form a respective plurality of second holes in at least one of the second cut surfaces that are aligned with the plurality of openings;
at least one first implant device including a plurality of pegs configured to be inserted in the plurality of first holes to securely mount the first implant device to the first cut surfaces of the tibial plateau; and
at least one second implant device including a plurality of pegs configured to be inserted in the plurality of second holes to securely mount the second implant device to the second cut surfaces of the medial condyle.

2. The system according to claim 1, wherein the at least one first cutting device comprises:
a tibial resection guide configured to be mounted to the tibia and defining a slot to slidably receive a cutting instrument to form one of the first cut surfaces; and
a tibial stylus configured to be mounted to the tibial resection guide and including a tip configured to define a predefined distance between the tip and the slot when mounted to the tibial resection guide.

3. The system according to claim 2, further comprising an extramedullary instrument including:
a clamp defining a slot, wherein the clamp is configured to be secured to a lower leg of the mammal below the knee; and
a rod configured to pass through the slot, wherein the rod has a first end and a second end opposite the first end, wherein the second end is received within a recess defined by the tibial resection guide.

4. The system according to claim 2,
wherein the tibial resection guide defines a plurality of cut predictors on an upper surface of the tibial resection guide; and
wherein when the tibial resection guide is mounted to the tibia at least one of the cut predictors is aligned with a region of the tibia such that a most lateral portion of the slot is no closer than a threshold distance from a medial aspect of an intercondylar eminence of the tibia in a transverse plane.

5. The system according to claim 1, wherein the at least one second cutting device comprises:
a femoral resection guide configured to be mounted to the medial condyle to form one of the second cut surfaces of the medial condyle; and
a femoral finishing guide configured to be mounted to the second cut surface formed by the femoral resection guide, said femoral finishing guide configured to form a plurality of the second cut surfaces.

6. The system according to claim 5,
wherein the femoral resection guide includes an upper surface that is shaped to match a shape of the medial condyle;
wherein the femoral resection guide defines a pin hole configured to receive a pin to be inserted in a most proximal edge of the medial condyle to secure the femoral resection guide to the medial condyle; and
wherein the femoral resection guide defines a cutting slot to slidably receive a cutting instrument to form the one of the second cut surfaces.

7. The system according to claim 6, wherein when the femoral resection guide is configured such that upon securing the femoral resection guide to the medial condyle, an angle between a longitudinal axis of the tibia and a longitudinal axis of the femur is within a predetermined range.

8. The system according to claim 7, wherein the predetermined range is between about 105 degrees and about 115 degrees.

9. The system according to claim 6, further comprising a femoral resection guide wedge configured to be positioned between a base of the femoral resection guide and the first cut surface in the tibial plateau to prevent a medial compartment of the knee from collapsing.

10. The system according to claim 9, wherein the system comprises a plurality of femoral resection guide wedges each having a different thickness along a longitudinal axis of the tibia when positioned on the first cut surface in the tibia plateau.

11. The system according to claim 5, wherein the femoral finishing guide defines a pair of slots that are oriented at different angles and configured to receive a cutting instrument to form the plurality of the second cut surfaces in the medial condyle.

12. The system according to claim 11, wherein a first slot of the pair of slots forms a first angle relative to a flat base of the femoral finishing guide and a second slot of the pair of slots forms a second angle relative to the flat base, wherein the first angle is in a range from about 60 degrees to about 70 degrees and the second angle is in a range from about 15 degrees to about 25 degrees.

13. The system according to claim 12, wherein the femoral finishing guide includes a flat surface adjacent to the pair of slots such that upon positioning the flat surface against the second cut surface formed by the femoral resection guide, the pair of slots are aligned with the medial condyle to form the plurality of second cut surfaces with the cutting instrument.

14. The system according to claim 13,
wherein the femoral finishing guide defines a pair of converging pin holes that are configured to receive respective pins to secure the femoral finishing guide to the second cut surface formed by the femoral resection guide;
wherein the femoral finishing guide includes a flat base connected to the flat surface;
and wherein the system further includes a femoral finishing guide wedge configured to be positioned between the flat base of the femoral finishing guide and the first cut surface in the tibial plateau to prevent a medial compartment of the knee from collapsing.

15. The system according to claim 1,
wherein the at least one first trial device is a tibial trial device with a profile based on a resected tibial plateau defined by the plurality of first cut surfaces; and
wherein the plurality of openings in the tibial trial device are peg holes that are oriented at a non-zero angle relative to a vertical direction that is orthogonal to a surface of the tibial trial device.

16. The system according to claim 1, wherein the at least one first implant device comprises:
a tibial baseplate defining a cavity and with the plurality of pegs that are oriented at a non-orthogonal angle relative to the tibial baseplate;
a tibial insert configured to be inserted within the cavity of the tibial baseplate.

17. The system according to claim 1, wherein the at least one second implant device comprises:
an inner surface that defines a plurality of surfaces that are angled relative to each other and wherein the plurality of pegs extend from at least one of the surfaces;
an outer surface that has an arcuate shape based on an arcuate shape of the medial condyle removed by the formation of the plurality of second cut surfaces.

18. The system according to claim 1, wherein the mammal is a horse and the system includes:
a plurality of the second cutting devices having different dimensions based on respective dimensions of the medial condyle of the horse;
a plurality of the first trial devices having different dimensions based on respective different dimensions of the tibia of the horse;
a plurality of the second trial devices having different dimensions based on respective different dimensions of the femur of the horse;
a plurality of the first implant devices having different dimensions based on respective different dimensions of the tibia of the horse; and
a plurality of the second implant devices having different dimensions based on respective different dimensions of the femur of the horse.

19. A method for performing a unicompartmental knee arthroplasty in a knee of a mammal, comprising:
cutting, with a first cutting device, a portion of a plateau of a tibia to form a plurality of first cut surfaces in the tibial plateau;
cutting, with a second cutting device, a portion of a medial condyle of a femur to form a plurality of second cut surfaces of the medial condyle;
aligning a plurality of openings in a first trial device with the first cut surfaces in the tibial plateau;
forming a respective plurality of first holes in the first cut surfaces based on passing a drill through the plurality of openings in the first trial device;
aligning a plurality of openings in a second trial device with the plurality of second cut surfaces of the medial condyle;
forming a respective plurality of second holes in the second cut surfaces based on passing a drill through the plurality of openings in the second trial device;
mounting a first implant device to the first cut surfaces of the tibial plateau based on inserting a plurality of pegs of the first implant device in the plurality of first holes; and
mounting a second implant device to the second cut surfaces of the medial condyle based on inserting a plurality of pegs of the second implant device in the plurality of second holes.

20. The method according to claim 19, wherein the cutting with the first cutting device comprises:
mounting a tibial resection guide to the tibia, wherein the tibial resection guide defines a slot to slidably receive a cutting instrument; and
cutting the tibia based on moving the cutting instrument along the slot to form one of the first cutting surfaces in the plateau of the tibia.

21. The method according to claim 20, wherein the cutting with the first cutting device further comprises:
mounting a tibial stylus to the tibial resection guide, wherein the tibial stylus includes a tip to define a predefined distance in a vertical direction in a coronal plane between the tip and the slot of the tibial resection guide; and
positioning the tip of the tibial stylus adjacent the plateau of the tibia such that the predefined distance in the vertical direction in the coronal plane is defined between the plateau of the tibia and the slot of the tibial resection guide; and
wherein the cutting step comprises removing a thickness of the tibia in the vertical direction in the coronal plane that does not exceed the predefined distance.

22. The method according to claim 20, wherein the cutting with the first cutting device further comprises:
securing a clamp of an extramedullary instrument to a lower leg of the mammal below the knee;
varying a position of a rod slidably received within a slot defined by the clamp, wherein the rod includes a first end and a second end opposite to the first end, wherein the second end is received within a recess of the tibial resection guide;
wherein an angle of the tibial resection guide relative to a longitudinal axis of the tibia within a sagittal plane is varied based on the varying step until the tibial resection guide is aligned with the plateau of the tibia in the sagittal plane.

23. The method according to claim 22, wherein the varying step is performed until the tibial resection guide is aligned with the plateau of the tibia in the sagittal plane and wherein the angle is between about 90 degrees and about 110 degrees.

24. The method according to claim 20,
wherein the mounting the tibial resection guide to the tibia comprises aligning one of a plurality of cut predictors on an upper surface of the tibial resection guide with a region of the tibia such that a most lateral portion of the slot is no closer than a threshold distance from a medial aspect of an intercondylar eminence of the tibia in a traverse plane; and
wherein the one of the first cutting surfaces formed by the cutting the tibia step is not closer than the threshold distance from the medial aspect of the intercondylar eminence of the tibia in the traverse plane.

25. The method according to claim 19, wherein the cutting with the second cutting device comprises:
mounting a femoral resection guide to the medial condyle to form one of the second cut surfaces of the medial condyle; and
mounting a femoral finishing guide to the second cut surface formed by the femoral resection guide to form a plurality of the second cut surfaces.

26. The method according to claim 25, wherein the mounting the femoral resection guide compriscs:
contacting an upper surface of the femoral resection guide with a surface of the medial condyle;
securing the femoral resection guide to the medial condyle based on inserting a pin through a pin hole defined by the femoral resection guide and into a most proximal edge of the medial condyle; and
wherein the cutting with the second cutting device comprises moving a cutting instrument within a cutting slot defined by the femoral resection guide to form one of the second cut surfaces.

27. The method according to claim 26, wherein when the cutting slot defines an angle relative to a longitudinal axis of the femur within a predetermined range.

28. The method according to claim 27, wherein the predetermined range is between about 105 degrees and about 115 degrees.

29. The method according to claim 26, wherein the mounting the femoral resection guide further comprises positioning a femoral resection guide wedge between a base of the femoral resection guide and the first cut surface in the tibial plateau to prevent a medial compartment of the knee from collapsing.

30. The method according to claim 29, wherein the method further comprising selecting the femoral resection guide wedge among a plurality of femoral resection guide wedges of different thickness along a longitudinal axis of the tibia when positioned on the first cut surface in the tibial plateau, wherein the femoral resection guide wedge is selected among the plurality of femoral resection guide wedges based on a dimension of a gap along the longitudinal axis of the tibia between the base of the femoral resection guide and the first cut surface.

31. The method according to claim 25, wherein the femoral finishing guide defines a pair of slots that are oriented at different angles and wherein the cutting with the second cutting device comprises moving a cutting instrument within each of the pair of slots to form the plurality of the second cut surfaces in the medial condyle.

32. The method according to claim 31, wherein the mounting the femoral finishing guide comprises positioning a flat surface of the femoral finishing guide against the second cut surface formed by the femoral resection guide such that the pair of slots are aligned with the medial condyle to form the plurality of the second cut surfaces based on the moving step.

33. The method according to claim 32, further comprising:
receiving respective pins in a pair of converging pin holes defined by the femoral finishing guide to secure the femoral finishing guide to the second cut surface formed by the femoral resection guide; and
positioning a femoral finishing guide wedge between a flat base of the femoral finishing guide and the first cut surface in the tibial plateau to prevent a medial compartment of the knee from collapsing.

34. The method according to claim 33, wherein the method further comprising selecting the femoral finishing guide wedge among a plurality of femoral finishing guide wedges of different thickness along a longitudinal axis of the tibia when positioned on the first cut surface in the tibial plateau, wherein the femoral finishing guide wedge is selected among the plurality of femoral finishing guide wedges based on a dimension of a gap along the longitudinal axis of the tibia between the flat base of the femoral finishing guide and the first cut surface.

35. The method according to claim 19, wherein the mounting the first implant device comprises:
inserting the plurality of pegs of a tibial baseplate defining a cavity in the plurality of first holes, wherein the tibial baseplate defines a cavity and wherein the plurality of pegs are oriented at a non-orthogonal angle relative to the tibial baseplate; and
inserting a tibial insert within the cavity of the tibial baseplate.

36. The method according to claim 19, wherein the plurality of second cut surfaces is three second cut surfaces that are angled relative to each other;
wherein the aligning the plurality of openings in the second trial device comprises aligning the plurality of openings with two of the three second cut surfaces; and
wherein the forming the respective plurality of second holes comprises passing the drill through a respective hole of the plurality of second holes in two of the three second cut surfaces.

37. The method according to claim 19, wherein the mounting the second implant device comprises:
inserting the plurality of pegs that extend from an inner surface of the second implant device that defines a plurality of surfaces that are angled relative to each other, wherein the plurality of pegs extend from at least one of the surfaces; and
wherein an outer surface of second implant device has an arcuate shape based on an arcuate shape of the medial condyle removed by the formation of the plurality of second cut surfaces.
